SEQUENCE LISTING (86 sequences)

SEQ ID NO: 1 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 2 - protein, 710 aa, Homo sapiens
MKLVFLVLLFLGALGLCLAGRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 3 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 4 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 5 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 6 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 7 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 8 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 9 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 10 - protein, 434 aa, Homo sapiens
MCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 11 - protein, 383 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTGPFSVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRNS

SEQ ID NO: 12 - protein, 114 aa, Homo sapiens
MCIAEIDLIPRDRPFVCAPSSKTGSVTTTYCCNQDHCNKIELPTTGLPLLVQRTIARTIVLQESIGKGRFGEVWRGKWRGEEVAVKIFSSREERSWFREAEIYQTVMLRHENIL

SEQ ID NO: 13 - protein, 118 aa, Homo sapiens
MCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTS

SEQ ID NO: 14 - protein, 126 aa, Homo sapiens
MCIAEIDLIPRDRPFVCAPSSKTGSVTTTYCCNQDHCNKIELPTTGPFSVKSSPGLGPVELAAVIAGPVCFVCISLMLMVYICHNRTVIHHRVPNEEDPSLDRPFISEGTTLKDLIYDMTTSGSGS

SEQ ID NO: 15 - protein, 122 aa, Homo sapiens
MCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGS

SEQ ID NO: 16 - protein, 114 aa, Homo sapiens
MCIAEIDLIPRDRPFVCAPSSKTGSVTTTYCCNQDHCNKIELPTTGPFSVKSSPGLGPVELAAVIAGPVCFVCISLMLMVYICHNRTVIHHRVPNEEDPSLDRPFISEGTTLKD

SEQ ID NO: 17 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 18 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 19 - protein, 592 aa, Homo sapiens; identical to SEQ ID NO: 17

SEQ ID NO: 20 - protein, 200 aa, Homo sapiens
MALWMRLLPLLALLALWGPDPAAAFVNQHLCGSHLVEALYLVCGERGFFYTPKTRREAEDLQASALSLSSSTSTWPEGLDATARAPPALVVTANIGQAGGSSSRQFRQRALGTSDSPVLFIHCPGAAGTAQGLEYRGRRVTTELVWEEVDSSPQPQGSESLPAQPPAQPAPQPEPQQAREPSPEVSCCGLWPRRPQRSQN

SEQ ID NO: 21 - protein, 503 aa, Homo sapiens
MTLGSPRKGLLMLLMALVTQGDPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLALILGPVLALLALVALGVLGLWHVRRRQEKQRGLHSELGESSLILKASEQGDSMLGDLLDSDCTTGSGSGLPFLVQRTVARQVALVECVGKGRYGEVWRGLWHGESVAVKIFSSRDEQSWFRETEIYNTVLLRHDNILGFIASDMTSRNSSTQLWLITHYHEHGSLYDFLQRQTLEPHLALRLAVSAACGLAHLHVEIFGTQGKPAIAHRDFKSRNVLVKSNLQCCIADLGLAVMHSQGSDYLDIGNNPRVGTKRYMAPEVLDEQIRTDCFESYKWTDIWAFGLVLWEIARRTIVNGIVEDYRPPFYDVVPNDPSFEDMKKVVCVDQQTPTIPNRLAADPVLSGLAQMMRECWYPNPSARLTALRIKKTLQKISNSPEKPKVIQ

SEQ ID NO: 22 - protein, 658 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA

SEQ ID NO: 23 - protein, 211 aa, Homo sapiens [sequence omitted]

SEQ ID NO: 24 - protein, 196 aa, Homo sapiens
MRTLACLLLLGCGYLAHVLAEEAEIPREVIERLARSQIHSIRDLQRLLEIDSVGSEDSLDTSLRAHGVHATKHVPEKRPLPIRRKRSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTDVR

SEQ ID NO: 25 - protein, 345 aa, Homo sapiens
MSLFGLLLLTSALAGQRQGTQAESNLSSKFQFSSNKEQNGVQDPQHERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIVMPQFTEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 26 - protein, 370 aa, Homo sapiens
MHRLIFVYTLICANFCSCRDTSATPQSASIKALRNANLRRDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 27 - protein, 1089 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 28 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 29 - protein, 194 aa, Homo sapiens
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 30 - protein, 208 aa, Homo sapiens
MWKWILTHCASAFPHLPGCCCCCFLLLFLVSSVPVTCQALGQDMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 31 - protein, 155 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 32 - protein, 822 aa, Homo sapiens [sequence omitted]

SEQ ID NO: 33 - protein, 217 aa, Homo sapiens
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 34 - protein, 202 aa, Homo sapiens
MKVLAAGVVPLLLVLHWKHGAGSPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 35 - protein, 1097 aa, Homo sapiens
MMDIYVCLKRPSWMVDNKRMRTASNFQWLLSTFILLYLMNQVNSQKKGAPHDLKCVTNNLQVWNCSWKAPSGTGRGTDYEVCIENRSRSCYQLEKTSIKIPALSHGDYEITINSLHDFGSSTSKFTLNEQNVSLIPDTPEILNLSADFSTSTLYLKWNDRGSVFPHRSNVIWEIKVLRKESMELVKLVTHNTTLNGKDTLHHWSWASDMPLECAIHFVEIRCYIDNLHFSGLEEWSDWSPVKNISWIPDSQTKVFPQDKVILVGSDITFCCVSQEKVLSALIGHTNCPLIHLDGENVAIKIRNISVSASSGTNVVFTTEDNIFGTVIFAGYPPDTPQQLNCETHDLKEIICSWNPGRVTALVGPRATSYTLVESFSGKYVRLKRAEAPTNESYQLLFQMLPNQEIYNFTLNAHNPLGRSQSTILVNITEKVYPHTPTSFKVKDINSTAVKLSWHLPGNFAKINFLCEIEIKKSNSVQEQRNVTIKGVENSSYLVALDKLNPYTLYTFRIRCSTETFWKWSKWSNKKQHLTTEASPSKGPDTWREWSSDGKNLIIYWKPLPINEANGKILSYNVSCSSDEETQSLSEIPDPQHKAEIRLDKNDYIISVVAKNSVGSSPPSKIASMEIPNDDLKIEQVVGMGKGILLTWHYDPNMTCDYVIKWCNSSRSEPCLMDWRKVPSNSTETVIESDEFRPGIRYNFFLYGCRNQGYQLLRSMIGYIEELAPIVAPNFTVEDTSADSILVKWEDIPVEELRGFLRGYLFYFGKGERDTSKMRVLESGRSDIKVKNITDISQKTLRIADLQGKTSYHLVLRAYTDGGVGPEKSMYVVTKENSVGLIIAILIPVAVAVIVGVVTSILCYRKREWIKETFYPDIPNPENCKALQFQKSVCEGSSALKTLEMNPCTPNNVEVLETRSAFPKIEDTEIISPVAERPEDRSDAEPENHVVVSYCPPIIEEEIPNPAADEAGGTAQVIYIDVQSMYQPQAKPEEEQENDPVGGAGYKPQMHLPINSTVEDIAAEEDLDKTAGYRPQANVNTWNLVSPDSPRSIDSNSEIVSFGSPCSINSRQFLIPPKDEDSPKSNGGGWSFTNFFQNKPND

SEQ ID NO: 36 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 37 - protein, 918 aa, Homo sapiens
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 38 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 39 - protein, 777 aa, Homo sapiens
MDSKESLTPGREENPSSVLAQERGDVMDFYKTLRGGATVKVSASSPSLAVASQSDSKQRRLLVDFPKGSVSNAQQPDLSKAVSLSMGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 40 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 41 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 42 - protein, 269 aa, Homo sapiens; identical to SEQ ID NO: 41

SEQ ID NO: 43 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 44 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 45 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 46 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 47 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 48 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 49 - protein, 221 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRHSPGRQSPDMPGDFRADAPSFLPPRRSLPMLFRMEWGCALTGSQSAVWPSSPVPEEIPRMHPGRNGKKQQRKPLREKMKPERCGDAVPRR

SEQ ID NO: 50 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 51 - protein, 93 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 52 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 53 - protein, 193 aa, Homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 54 - protein, 508 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 55 - protein, 153 aa, Homo sapiens [sequence omitted]

SEQ ID NO: 56 - protein, 825 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 57 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 58 - protein, 288 aa, Homo sapiens; identical to SEQ ID NO: 5

SEQ ID NO: 59 - protein, 658 aa, Homo sapiens; identical to SEQ ID NO: 22

SEQ ID NO: 60 - protein, 798 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 61 - protein, 742 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 62 - protein, 574 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 63 - protein, 161 aa, Homo sapiens
MNLAISIALLLTVLQVSRGQKVTSLTACLVDQSLRLDCRHENTSSSPIQYEFSLTRETKKHVLFGTVGVPEHTYRSRTNFTSKYNMKVLYLSAFTSKDEGTYTCALHHSGHSPPISSQNVTVLRDKLVKCEGISLLAQNTSWLLLLLLSLSLLQATDFMSL

SEQ ID NO: 64 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 65 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 66 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 67 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 68 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 69 - protein, 385 aa, Homo sapiens
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLGEDPYYTENGGGQGYSSGPGTSPEAQGKASVNRGAQENGTGQATSRNGHSARQHVVADTEL

SEQ ID NO: 70 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 71 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 72 - protein, 556 aa, Homo sapiens [sequence omitted]

SEQ ID NO: 73 - protein, 495 aa, Homo sapiens
MPMGSLQPLATLYLLGMLVASCLGRLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPAGLAAGTVASIILALVLLVVLLVVCGPLAYKKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 74 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 75 - protein, 1152 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 76 - protein, 375 aa, Homo sapiens
MERASCLLLLLLPLVHVSATTPEPCELDDEDFRCVCNFSEPQPDWSEAFQCVSAVEVEIHAGGLNLEPFLKRVDADADPRQYADTVKALRVRRLTVGAAQVPAQLLVGALRVLAYSRLKELTLEDLKITGTMPPLPLEATGLALSSLRLRNVSWATGRSWLAELQQWLKPGLKVLSIAQAHSPAFSCEQVRAFPALTSLDLSDNPGLGERGLMAALCPHKFPAIQNLALRNTGMETPTGVCAALAAAGVQPHSLDLSHNSLRATVNPSAPRCMWSSALNSLNLSFAGLEQVPKGLPAKLRVLDLSCNRLNRAPQPDELPEVDNLTLDGNPFLVPGTALPHEGSMNSGVVPACARSTLSVGVSGTLVLLQGARGFA

SEQ ID NO: 77 - protein, 223 aa, Homo sapiens
MPGGRRALQTPPATIFLLLISAAALGPCCQALWVDWGPPSVTVSVGDKVSLPCRHNSNSTNITWWRVPQGNSTWPPVMDLSELGPKGELIIQQVNKSHRGMYRCEVNEGQRIQRSCGTYLRVRDPLPRPFLDMGEGTKNNIITAEGIILLICAVVPGTLLLFRKRWQNVKFGADVQDDYDDENLYEGLNLDDCSMYEDISRGLQGTYQDVGSLHIGDVQLEKP

SEQ ID NO: 78 - protein, 296 aa, Homo sapiens
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM

SEQ ID NO: 79 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 80 - protein, 266 aa, Homo sapiens
MVCLKLPGGSCMTALTVTLMVLSSPLALSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 81 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 82 - protein, 146 aa, Homo sapiens [sequence omitted]

SEQ ID NO: 83 - protein, 1165 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 84 - protein, 210 aa, Homo sapiens
MGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 85 - protein, 155 aa, Homo sapiens [sequence omitted]

SEQ ID NO: 86 - protein, 196 aa, Homo sapiens
MGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

(19) United States
(12) Patent Application Publication
(10) Pub. No.: US 2021/0115395 A1
(43) Pub. Date: Apr. 22, 2021

(54) METHODS FOR EXPANDING ADHERENT STROMAL CELLS AND CELLS OBTAINED THEREBY

(71) Applicant: PLURISTEM LTD., Haifa (IL)

(72) Inventors: Lior RAVIV, Kfar Monash (IL); David WILDAWER, Haifa (IL); Eva Bjerge ZINO, Atlit (IL)

(73) Assignee: PLURISTEM LTD., Haifa (IL)

(21) Appl. No.: 17/040,993

(22) PCT Filed: Mar. 28, 2019
(86) PCT No.: PCT/IB2019/052569

§ 371 Date: Sep. 24, 2020

Related U.S. Application Data
(60) Provisional application No. 62/648,960, filed on Mar. 28, 2018.

(51) Int. Cl. C12N 5/073; A61K 35/50

(52) U.S. Cl. CPC C12N 5/0605; A61K 35/50; C12N 2533/52; C12N 2501/25; C12N 2501/2304; C12N 2513/00; C12N 2500/92; C12N 2501/113; C12N 2501/115; C12N 2501/15; C12N 2501/135; C12N 2501/2301

(57) ABSTRACT

Disclosed herein are methods of expanding adherent stromal cells, including, inter alia, multi-step methods. Also disclosed are cells produced by the methods, which may be adherent stromal cells, for example placental adherent stromal cells. Further disclosed are pharmaceutical compositions comprising the cells. Additionally, methods of producing and utilizing the compositions, for example for therapeutic uses, are described.

### FIELD

Disclosed herein are methods of expanding placental adherent stromal cells.

### BACKGROUND

Worldwide, cell culture in both academia and industry is highly dependent on various sera, such as fetal bovine serum (FBS). Increasing demand and limited availability has caused the price of FBS to increase several-fold in recent years. In addition, collection of FBS is the subject of ethical controversy (Fang C Y et al), and it creates significant regulatory concerns. Despite the huge ethical and financial costs of FBS use, and regulatory concerns, alternatives have not been widely adopted. Chemically defined alternatives to serum need to be empirically tested in each potential cell culture application.

### SUMMARY

As provided herein, ASC (e.g. placental ASC) are expanded in a serum-free medium. The resulting cells exhibit a unique set of characteristics and properties that are not believed to have any counterpart, either in nature or in previously-known artificially-produced cell compositions. They are, in some embodiments, induced as a result of the described ex-vivo expansion steps to produce or secrete elevated amounts of therapeutic factors. In still other embodiments, they are suitable for use in tissues distant from their site of administration, or, in other embodiments, when administered systemically.

In certain embodiments, the ASC are cultured on a 2-dimensional (2D) substrate, a 3-dimensional (3D) substrate, or a 2D substrate, followed by a 3D substrate. Non-limiting examples of 2D and 3D substrates are provided in the Detailed Description and Examples.

The terms “two-dimensional culture”, “2D culture”, and “two-dimensional [or 2D] substrate” refer to a culture in which the cells are exposed to conditions that are compatible with cell growth and allow the cells to grow in a monolayer, which is referred to as a “two-dimensional culture apparatus”. Such apparatuses will typically have flat growth surfaces, in some embodiments comprising an adherent material, which may be planar or curved. Non-limiting examples of apparatuses for 2D culture are cell culture dishes and plates. Included in this definition are multi-layer trays, such as Cell Factory™, manufactured by Nunc™, provided that each layer supports monolayer culture. It will be appreciated that even in 2D apparatuses, cells can grow over one another when allowed to become over-confluent. This does not affect the classification of the apparatus as “two-dimensional”.

The terms “three-dimensional culture”, “3D culture”, and “three-dimensional [or 3D] substrate” refer to a culture in which the cells are exposed to conditions that are compatible with cell growth and allow the cells to grow in a 3D orientation (for example, outside of the plane of a monolayer) relative to one another. The term “three-dimensional [or 3D] culture apparatus” refers to an apparatus for culturing cells under conditions that are compatible with cell growth and allow the cells to grow in a 3D orientation relative to one another. Such apparatuses will typically have a 3D growth surface, in some embodiments comprising an adherent material. Certain, non-limiting embodiments of 3D culturing conditions suitable for expansion of ASC are described in PCT Application Publ. No. WO/2007/108003 and WO 2010/026575, the contents of which are incorporated by reference as if fully set forth herein.

In general, reference to cell “growth” and “expansion” may be used interchangeably herein. In some embodiments, the described cell expansion is differentiation-less expansion, which may apply, in certain embodiments, to any of the methods and compositions described herein.

In certain embodiments, the cells that are subject to expansion are mesenchymal-like ASC, which exhibit a marker pattern similar to mesenchymal stromal cells (MSC), but do not readily differentiate into osteocytes, under conditions where “classical” MSC would differentiate into osteocytes. In other embodiments, the cells exhibit a marker pattern similar to MSC, but do not readily differentiate into adipocytes, under conditions where MSC would differentiate into adipocytes. In still other embodiments, the cells exhibit a marker pattern similar to MSC, but do not differentiate into either osteocytes or adipocytes, under conditions where MSC would differentiate into osteocytes or adipocytes, respectively. The MSC used for comparison in these assays are, in some embodiments, MSC that have been harvested from bone marrow (BM) and cultured under 2D conditions. In other embodiments, the MSC used for comparison have been harvested from BM and cultured in 2D culture, followed by 3D culture. In more particular embodiments, the described mesenchymal-like ASC are placental cells of maternal origin. In alternative embodiments, the mesenchymal-like ASC are placental cells of fetal origin. In still other embodiments, the mesenchymal-like ASC are a mixture of maternal and fetal cells. In a non-limiting embodiment, a mixture of maternal and fetal placental cells can be obtained by mincing whole placenta or in other embodiments a portion thereof; or, in still other embodiments, whole placenta, apart from the amnion, chorion, and/or umbilical cord.

Except where otherwise indicated, all ranges mentioned herein are inclusive.

Except where otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a diagram of a bioreactor that can be used to prepare the cells.

FIG. 2A is a perspective view of a carrier (or “3D body”), according to an exemplary embodiment. B is a perspective view of a carrier, according to another exemplary embodiment. C is a cross-sectional view of a carrier, according to an exemplary embodiment.

FIG. 3 is a graph depicting log of fold-change of protein expression, relative to a reference standard (vertical axis), of various proteins (horizontal axis) by ASC subjected to activation in a bioreactor. Black and striped bars show groups stimulated with concentrations of 25/25 and 10/10, respectively.

FIGS. 4A-B are graphs depicting log of fold-change of protein expression, relative to a reference standard (vertical axis), of various proteins (horizontal axis) by ASC subjected to activation for 24 hrs. (A) or 96 hrs. (B) in tissue culture flasks. For A, black, white and gray bars show groups stimulated with concentrations of 25/25, 10/10, and 5/5 respectively. For B, striped, white, and black show groups stimulated with concentrations of 25/25, 10/10, and 5/5.

FIG. 5 is a plot depicting percent survival (vertical axis) of mice subjected to irradiation and treated with vehicle or ASC activated with activating factors (batches 1-3) or serum (batches 4-5). Horizontal axis: days post-irradiation. Batches 3 and 4 had the same curves.

FIG. 6 is a plot depicting percent survival (vertical axis) of mice subjected to irradiation and treated with vehicle or ASC activated with activating factors (batches 1-3) or serum (batches 4-5). Horizontal axis: days post-irradiation. Batches 1 and 2 had the same curves.

### DETAILED DESCRIPTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Provided herein are methods of incubating, expanding, and/or producing altered populations of adherent stromal cells (ASC), cells produced by the described methods, pharmaceutical compositions comprising the cells, and methods of producing and using the cells and compositions. ASC may be derived, for example, from placenta; adipose tissue; bone marrow (BM); peripheral blood; umbilical cord blood (UCB); synovial fluid; synovial membranes; spleen; thymus; mucosa (for example nasal mucosa); limbal stroma; ligaments, for example the periodontal ligament; scalp; hair follicles; testicles; embryonic yolk sac; and amniotic fluid, all of which are known to include ASC. In certain embodiments, the source of the ASC is a non-fetal source, for example either (a) maternal cells from the placenta; or (b) somatic tissue from a pediatric or adult donor, for example adipose tissue, BM, peripheral blood, UCB, synovial fluid, synovial membranes, and ligaments (a non-limiting example of which is the periodontal ligament). In some embodiments, the ASC are human ASC, while in other embodiments, they may be animal ASC. In particular embodiments, the ASC are derived from placental tissue, or are derived from adipose tissue.

In some embodiments, there is provided a method of expanding a population of ASC, comprising: incubating the ASC population in a medium, wherein the medium contains less than 5% animal serum, thereby obtaining an expanded cell population.

In other embodiments, there is provided a method of expanding a population of ASC, comprising: a. incubating the ASC population in a first medium, wherein the first medium contains less than 5% animal serum, thereby obtaining a first expanded cell population; and b. incubating the first expanded cell population in a second medium, wherein the second medium also contains less than 5% animal serum, and wherein one or more activating components are added to the second medium. This second medium can also be referred to herein as an activating medium. In other embodiments, the first medium or the second medium, or in other embodiments both the first and second medium, is/are serum free. In still other embodiments, the first medium contains a first basal medium, with the addition of one or more growth factors, collective referred to as the “first expansion medium” (to which a small concentration of animal serum is optionally added); and the activating medium contains a second basal medium with the addition of one or more growth factors (the “second expansion medium”), to which activating component(s) are added. In more specific embodiments, the second expansion medium is identical to the first expansion medium; while in other embodiments, the second expansion medium differs from the first expansion medium in one or more components.

In certain embodiments, the aforementioned step of incubating the ASC population in a first medium is performed for at least 17 doublings, or in other embodiments at least 6, 8, 12, 15, or at least 18 doublings; or 12-30, 12-25, 15-30, 15-25, 16-25, 17-25, or 18-25 doublings.

In other embodiments, the ASC population is incubated in the aforementioned first medium for a defined number of passages, for example 2-3, or in other embodiments 1-4, 1-3, 1-2, or 2-4; or a defined number of population doublings, for example 4-7, or in other embodiments at least 4, at least 5, at least 6, at least 7, at least 8, 4-10, 4-9, 4-8, 5-10, 5-9, or 5-8. The cells are then cryopreserved, then subjected to additional culturing in the first medium. In some embodiments, the additional culturing in the first medium is performed for 6-10 population doublings, or in other embodiments at least 6, at least 7, at least 8, at least 9, at least 10, 6-20, 7-20, 8-20, 9-20, 10-20, 6-15, 7-15, 8-15, 9-15, or 10-15 population doublings. Alternatively, the additional culturing in the first medium is performed for 2-3 passages, or in other embodiments at least 1, at least 2, at least 3, 1-5, 1-4, 1-3, 2-5, or 2-4 passages.

In still other embodiments, the step of incubating the first expanded cell population in a second medium is performed for a defined number of total passages, for example 3-5 passages, or in other embodiments 1-4, 1-3, 2-3, 2-5, or 2-4; or a defined number of total population doublings, for example 12-20, or in other embodiments 12-15, or in other embodiments 15-20, 12-18, 12-16, 14-20, or 14-18 doublings.

In other embodiments, the ASC population is incubated in the second medium for a defined number of days, for example 4-10, 5-10, 6-10, 4-9, 4-8, 4-7, 5-9, 5-8, 5-7, 6-10, 6-9, or 6-8; or a defined number of population doublings, for example at least 3, at least 4, at least 5, at least 6, 3-10, 3-9, 3-8, 4-10, 4-9, or 4-8. The cells are then subjected to additional culturing in the second medium in a bioreactor. In some embodiments, the bioreactor culturing is performed for at least 4, at least 5, at least 6, at least 7, at least 8, at least 9, 4-10, 4-9, 4-8, 5-10, 5-9, 5-8, 6-10, 6-9, or 6-8 population doublings; or, in other embodiments, for at least 4, at least 5, at least 6, at least 7, 4-15, 4-12, 4-10, 4-9, 4-8, 4-7, 4-15, 5-12, 5-10, 5-9, 5-8, 5-7, 6-15, 6-12, 6-10, 6-9, 6-8, or 6-7 days. In certain embodiments, the bioreactor contains 3D carriers, on which the cells are cultured.

In certain embodiments, the aforementioned two-stage incubation is preceded by culturing in a medium containing over 5% animal serum (e.g. as described herein). In general, for such embodiments, the nomenclature of the aforementioned steps is retained. Thus, the first medium (containing less than 5% animal serum) still retains its designation as the “first medium”, and the activating medium retains its designation as the “second [or activating] medium”.

In other embodiments, there is provided a method of expanding a population of ASC, comprising: a. incubating the ASC population in a serum-free medium (SFM), or in other embodiments a serum-poor medium (SPM), thereby obtaining a first expanded cell population; and b. incubating the first expanded cell population in a second medium, wherein the second medium contains at least 10% animal serum. (In embodiments in which incubation in the first medium is preceded by culturing in a medium containing over 5% animal serum [as described herein], the SFM or SPM still retains its designation as the “first medium”, and the following medium [containing at least 10% animal serum] retains its designation as the “second medium”). The second medium, in some embodiments, contains an animal serum content of 10-25%, 11-25%, 12-25%, 13-25%, 14-25%, 15-25%, 10-24%, 10-23%, 10-22%, 10-21%, 10-20%, 11-19%, 12-18%, 13-17%, 16-24%, 17-23%, or 18-22%. In other embodiments, the second medium contains at least 11%, 12%, 13%, 14%, 15%, 16%, 17%, 18%, 19%, or 20% animal serum. In certain embodiments, the second medium does not contain added growth factors, other than those present in the animal serum added thereto.

Those skilled in the art will appreciate, in light of the present disclosure, that the ASC that is used in the described methods has been extracted, in some embodiments, from the placenta, from adipose tissue, or from other sources, e.g. as mentioned herein.

SFM, as used herein, indicates a serum-free medium; and SPM indicates a serum-poor medium. In certain embodiments, the SPM contains less than 5% animal serum. The described SFM may be, in more specific embodiments, serum-replacement medium (described hereinbelow). In certain embodiments, the SPM contains less than 4%; less than 3%; less than 2%; less than 1%; less than 0.5%; less than 0.3%; less than 0.2%; or less than 0.1% animal serum. In other embodiments, the SFM/SPM is a defined medium to which a low concentration of serum (SPM); or in other embodiments, no serum (SFM), has been added. In other embodiments, the SFM/SPM contains a basal medium, with the addition of one or more growth factors. In certain embodiments, the growth factors, individually or collectively, induce cell expansion in culture. In other embodiments, the growth factors, individually collectively, induce cell expansion in culture without differentiation.

Those skilled in the art will appreciate that reference herein to animal serum includes serum from a variety of species, provided that the serum stimulates expansion of the described ASC population. In certain embodiments, the serum is mammalian serum, non-limiting examples of which are human serum, bovine serum (e.g. fetal bovine serum and calf bovine serum), equine serum, goat serum, and porcine serum.

The basal medium or expansion medium used in the described methods and compositions can be, in certain embodiments, an SRM described herein, non-limiting examples of which are MSC Nutristem® XF basal medium, Stempro® SFM, Stempro® SFM-XF, PPRF-msc6, D-hESF10, TheraPEAK™ MSCGM-CDTM, DMEM/F-12, and MesenCult-XF.

In some embodiments, the basal medium is used alone, while in other embodiments, growth factors, activating components, and/or cytokines are added (analogous to MSC Nutristem® XF full medium).

In other embodiments, the described expansion medium includes a standard basal medium, or a functional variant thereof, with the addition of one or more growth factors. Non-limiting examples of standard basal media useful in cell culturing include Minimum Essential Medium Eagle, ADC-1, LPM (Bovine Serum Albumin-free), F10(HAM), F12/DMEM, DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM; Dulbecco and Freeman), Yamane, IMEM-20, Glasgow Modification Eagle Medium (GMEM), Leibovitz L-15 Medium, McCoy's 5A Medium, Medium M199 (M199E—with Earle's sale base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with non-essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153, Iscove's Modified Dulbecco's Medium (IMDM), MCDB201 (Sigma-Aldrich/Merck cat. no. M6770) and mixtures thereof in any proportions.

In certain embodiments, DMEM or a DMEM-based medium is used. Solely by way of exemplification, F12/DMEM is a mixture of DMEM and F12 nutrient mixture. An exemplary F12 nutrient mixture may contain effective concentrations of inorganic salts, e.g. Calcium chloride (CaCl₂) (44), Cupric sulfate (CuSO₄-5H₂O) (0.00125), Ferrous sulfate (FeSO₄-7H₂O) (0.83), Potassium chloride (KCl) (220), Magnesium Chloride (57), Sodium chloride (NaCl) (7600), Sodium hydrogen phosphate (Na2HPO4) (140), and Zinc sulfate (ZnSO₄-7H₂O) (0.86); amino acids, e.g. L-Alanine (8.9), L-Arginine hydrochloride (210), L-Asparagine (15), L-Aspartic acid (13), L-Cystine hydrochloride monohydrate (35), L-Glutamic acid (14), L-Glutamine (146), Glycine (7.5), L-Histidine-HCl—H₂O (23), L-Isoleucine (3.9), L-Leucine (13.10), L-Ly sine hydrochloride (36.5), L-Methionine (4.5), L-Phenylalanine (5.0), L-Proline (34.5), L-Serine (10.50), L-Threonine (12), L-Tryptophan (2.0), L-Tyrosine disodium (7.8), and L-Valine (11.7); vitamins, e.g. Biotin (0.007), Choline chloride (14.0), Folic acid (1.3), i-Inositol (18), Nicotinamide (0.037), Riboflavin (0.038), Thiamine hydrochloride (0.34), Vitamin B12 (1.36), D-Calcium pantothenate (0.24), and Pyridoxine hydrochloride (0.062); and other compounds, e.g. D-Glucose (1800), Hypoxanthine (5.4), Linoleic Acid (0.084), Lipoic Acid (0.21), Putrescine Dihydrochloride (0.16), Sodium Pyruvate (110), and Thymidine (0.73). Optional additional components include, e.g. a pH buffer (e.g. HEPES or bicarbonate buffer), Phenol red, Calcium Pantothenate, Niacinamide, Ammonium Molybdate, Ammonium Metavandate, Manganese Sulfate, Nickel Chloride, Sodium Bicarbonate, Sodium Metasilicate, Sodium Selenite, Stannous Chloride, and lipids (e.g. Arachidonic Acid, Linoleic Acid, Linolenic Acid, Myristic Acid, Oleic Acid, Palmitic Acid, and Stearic Acid). Suggested concentrations (in mg/L) are provided solely for the purposes of exemplification. Those skilled in the art will readily understand, given the information provided herein, that the concentrations of these components can be varied while remaining within the effect range of concentrations.

In other embodiments, the basal medium comprises one or more inorganic salts, which optionally collectively serve as a pH buffer; essential amino acids for mammalian cell culture; one or more vitamins (e.g. essential vitamins for mammalian cell culture); glucose, and one, or in other embodiments at least 2, at least 3, at least 4, at least 5, or all of Hypoxanthine, Linoleic Acid, Lipoic Acid, Putrescine Dihydrochloride, Sodium Pyruvate, and Thymidine. Essential components for mammalian cell culture known in the art, and are described, for example in Dulbecco and Freeman.

These and other useful media are available from GIBCO, Grand Island, N.Y., USA and Biological Industries, Bet HaEmek, Israel, among others. Those skilled in the art can readily identify functional variants of standard basal media, by verifying that the variants contain appropriate concentrations of the components necessary for cell expansion and vitality. Incubation with cells can optionally be used to verify the suitability of a particular formulation.

In certain embodiments, one or more factors that promote cell spreading and attachment are added to the basal medium. Non-limiting examples of such factors are Fibronectin and Vimentin, Collagen, and Laminin. The skilled person will appreciate that both modified and native forms of such factors may be used, provided that they maintain their adhesion-promoting properties. Alternatively, other Integrin ligands may be utilized, as will be appreciated by those skilled in the art, and as described, for example, in Docheva D et al. and the references cited therein.

Growth Factors

Growth factors are generally known in the art, and are described, inter alia in Ng et al, and the references cited therein.

In more specific embodiments, the growth factor(s) contained in the described SFM, SPM, and expansion media is selected from one or more of: an FGF (embodiments of which are mentioned herein), TGF-beta; Transferrin (e.g. serotransferrin or lactotransferrin; Uniprot accession nos. P02787 and P02788; SEQ ID Nos. 1-2); Insulin (Uniprot accession nos. P01308 and F8WCM5; SEQ ID Nos. 3 and 20); EGF (epidermal growth factor; proprotein: Uniprot accession no. P01133; SEQ ID Nos. 4 and 83); LIF; a glucocorticoid; and/or PDGF. In certain embodiments, the growth factors comprise 2 or more of bFGF (a.k.a. Fibroblast Growth Factor 2; Uniprot accession no. P09038; SEQ ID No. 5 and 84-86), TGF-beta, and PDGF (e.g. PDGF-BB). In still other embodiments, the factors comprise bFGF, TGF-beta, and PDGF (e.g. PDGF-BB). In yet other embodiments, the expansion medium used in the described methods consists of, or in other embodiments consists essentially of, a basal medium, with the addition of bFGF, TGF-beta, and PDGF (e.g. PDGF-BB); one or more fatty acids; Insulin; Transferrin; optionally one or more other carrier proteins; Selenium; one or more antioxidants; and one or more pH buffers. In still other embodiments, L-Glutamine is also present.

All Uniprot accession nos. in this document were accessed on Mar. 28, 2019.

TGF-beta, as used herein, may refer to, for example, TGF-beta-1, the proprotein of which is set forth in Uniprot accession no. P01137 (SEQ ID No. 6); TGF-beta-2 and TGF-beta-3, the proproteins of which are set forth in Uniprot accession nos. P61812 and P10600 (SEQ ID No. 7-8); and homodimers and mixed dimers of TGF-beta-1, TGF-beta-2, and TGF-beta-3 (Derynck and Budi, Heldin and Moustakas, and the references cited therein). Those skilled in the art will appreciate that non-human versions of TGF-beta proteins, as well as fragments, variations and mimetics thereof, may be used on human target cells, provided that they exhibit a significant portion of the activity of the human versions, when interacting with human cells. Relevant TGF-beta activities, in some embodiments, include stimulation of TGF-β receptor (TGFBR), which may, in some embodiments, contain two Type I TGFBR family subunits and two Type II TGFBR family subunits. Examples of Type I receptors include TGF-beta receptor type-1 (Uniprot accession no. P36897; SEQ ID No. 9), and its isoforms, as set forth in Uniprot entries B4DY26, F8W1R9, F8VXZ5, F8VVC4, F8WOK6, H0YHS4, and F8VRH6; SEQ ID Nos. 10-16). Examples of Type I receptors include TGF-beta receptor type-2 (Uniprot accession no. D2JYI1, P37173-1, and P37173-2; SEQ ID Nos. 17-19). In more specific embodiments, the activity includes binding a heterotetramer of 2 units each of TGF-beta receptor type-1 and TGF-beta receptor type-2. In yet other embodiments, the activity further includes binding to a heterotetramer of 2 units each of ALK-1 (Serine/threonine-protein kinase receptor R3; Uniprot Accession No. P37023; SEQ ID No. 21) and TGF-beta receptor type-2, optionally in conjunction with Endoglin (Uniprot Accession No. P17813; SEQ ID No. 22). In other embodiments, WNT-3 is used instead of TGF-beta. In certain embodiments, the TGF-beta protein is selected from TGF-beta-1 and TGF-beta-3, which act similarly.

PDGF, as used herein, may refer to platelet-derived growth factor, including any combination of subunits A and B (Uniprot accession nos. P04085-1 and P04085-2; and P01127, respectively; SEQ ID Nos. 23, 24, and 36); or, in other embodiments homodimers of C and D (Uniprot accession nos. Q9NRA1 and Q9GZP0, respectively; SEQ ID Nos. 25-26), each of which represents a separate embodiment. A non-limiting example of PDGF is PDGF-BB. In still other embodiments, a different PDGF isoform is used, which may be, in more specific embodiments, PDGF-AA, PDGF-AB, PDGF-CC, or PDGF-DD. In certain embodiments, a ligand of PDGFRα (Uniprot Accession No. P16234; SEQ ID No. 27) (e.g. PDGF-AA, PDGF-AB, PDGF-BB, and PDGF-CC); PDGFRβ (Uniprot Accession No. P09619; SEQ ID No. 28) (e.g. PDGF-BB and PDGF-DD); or PDGFRαβ (e.g. PDGF-AB, PDGF-BB, PDGF-CC, and PDGF-DD) is used. PDGF isoforms are known in the art, and are described, inter alia, in Kazlauskas A and the references cited therein. Those skilled in the art will appreciate that non-human versions of PDGF proteins, as well as fragments, variations and mimetics thereof, may be used on human target cells, provided that they exhibit a significant portion of the activity of the human versions, when interacting with human cells. Relevant PDGF activities include stimulation of PDGFRα, PDGFRβ, and/or PDGFRαβ, each of which represents a separate embodiment. In other embodiments, the PGDF is selected from PDGF-BB AND PDGF-AB, which act similarly.

The FGF (fibroblast growth factor) family includes a number of human and non-human proteins that are described in Imamura, Itoh N. et al, and the references cited therein. Non-limiting examples are anagen-promoting FGF's, e.g. bFGF, FGF7 (Uniprot Accession No. P21781; SEQ ID No. 29), FGF10 (Uniprot Accession No. 015520; SEQ ID No. 30), and FGF1 (Uniprot Accession No. P05230; SEQ ID No. 31). FGFs can be classified into paracrine FGFs, e.g. FGF1-10, 16-18, 20, and 22; intracrine FGFs, e.g. FGF11-14; and endocrine FGFs, e.g. FGF19, 21, and 23. Paracrine FGFs can be further classified into the following subfamilies: FGF 1/2, FGF 3/7/10/22, FGF 4/5/6, FGF 8/17/18, and FGF 9/16/20. Most are secreted proteins with cleavable N-terminal secreted signal peptides; however, FGF9, FGF16 and FGF20 have uncleaved bipartite secreted signal sequences (Itoh N. et al and references cited therein). Those skilled in the art will appreciate that non-human versions of FGFs, as well as fragments, variations and mimetics thereof, may be used on human target cells, provided that they exhibit a significant portion of the activity of the human versions, e.g. stimulation of Fibroblast growth factor receptor 1 (Uniprot Accession No. P11362; SEQ ID No. 32), when interacting with human cells (Coffin J D et al and references cited therein). In other embodiments, EGF is used in place of FGF. In still other embodiments, Somatotropin (Growth Hormone; Uniprot Accession No. P01241; SEQ ID No. 33) is used in place of FGF.

In more specific embodiments, the described FGF is selected from FGF1, FGF2, and chimeras thereof (a non-limiting example of which is FGFC [Imamura]).

LIF, as used herein, may refer to Leukemia Inhibitory Factor (Uniprot accession no. P15018; SEQ ID No. 34). LIF signals via a receptor containing LIFRβ (a.k.a. Leukemia inhibitory factor receptor Uniprot accession no. P42702; SEQ ID No. 35) and gp130 (Interleukin-6 receptor subunit beta; Uniprot accession no. P40189; SEQ ID No. 37). LIF proteins are known in the art, and are described, inter alia, in Nicola and Babon, Davis and Pennypacker, and the references cited therein. Those skilled in the art will appreciate that non-human versions of LIF proteins, as well as fragments, variations and mimetics thereof, may be used on human target cells, provided that they exhibit a significant portion of the activity of the human version, e.g. stimulation of the LIF receptor, when interacting with human cells. In other embodiments, IL-6 (Interleukin-6; Uniprot accession no. P05231; SEQ ID No. 38) is used instead of LIF. In still other embodiments, Somatotropin is used in place of FGF.

Glucocorticoid, as used herein, refer to the class of compounds that bind Glucocorticoid receptor (Uniprot accession no. P04150; SEQ ID No. 39). In certain embodiments, the glucocorticoid is selected from cortisones, dexamethasones, hydrocortisones, methylprednisolones, prednisolones and prednisones. Exemplary cortisones include 21-acetoxypregnenolone, alclometasone, algestone, amcinonide, beclomethasone, betamethasone, budesonide, chloroprednisone, ciclesonide, clobetasol, clobetasone, clocortolone, cloprednol, corticosterone, cortisone, cortivazol, deflazacort, desciclesonide, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolonc, tixocortol, triamcinolone, triamcinolone acetonide, triamcinolone benetonide, triamcinolone hexacetonide. Non-limiting examples of hydrocortisones (HC) are hydrocortisone (e.g. 11β,17α,21-trihydroxypregn-4-ene-3,20-dione) and salts thereof. Those skilled in the art will, appreciate, in light of the present disclosure, that derivatives, analogues, enantiomer forms, stereoisomers, anhydrides, acid addition salts, and base salts may substitute for the described glucocorticoid compounds, provided that they maintain the relevant biological activity.

In other embodiments, the growth factors comprise 2 or more of FGF (e.g. bFGF), TGF-beta, LIF, and a glucocorticoid. In other embodiments, the growth factors comprise 3 or more of FGF, TGF-beta, LIF, and a glucocorticoid. In still other embodiments, the factors comprise FGF, TGF-beta, LIF, and a glucocorticoid. In yet other embodiments, the expansion medium used in the described methods consists of, or in other embodiments consists essentially of, a basal medium, with the addition of FGF, TGF-beta, LIF, and a glucocorticoid; one or more fatty acids; Insulin; Transferrin; optionally one or more other carrier proteins; Selenium; one or more antioxidants; and one or more pH buffers. In still other embodiments, L-Glutamine is also present.

In other embodiments, a carrier protein is present. A non-limiting example of a carrier protein is albumin (e.g. human albumin, or, in other embodiments, albumin from another species, e.g. bovine albumin). Those skilled in the art will appreciate that the exact choice of a carrier protein is not critical to the disclosed methods and compositions. In more specific embodiments, the carrier protein is present at a concentration of 0.4-4 mg/mL, or, in other embodiments, 0.5-4, 0.5-3, 0.5-2.5, 0.5-2, 0.5-10, 0.5-8, 0.5-6, 0.5-5, 1-10, 1-5, 2-5, 1-8, 1-6, 2-10, 2-8, 2-6, 2-5, 3-10, 3-8, 3-6, or 3-5 mg/mL.

As mentioned, in certain embodiments of the described multi-stage cell expansion methods, the first and second expansion media independently comprise a basal medium with the addition of one or more growth factors. In various embodiments, the first and second expansion media may be the same (excluding the activating component(s) in the latter) or different from one another. All the embodiments described for the amounts (or lack) of animal sera, basal media, and growth factors apply independently to the first and second expansion media. In certain embodiments, the first and second expansion media comprise the same basal media. Alternatively or in addition, the two expansion media comprise at least 1, at least 2, at least 3, or at least 4 common growth factors. In other embodiments, the two expansion media contain the same set of growth factors.

Except where indicated otherwise, reference herein to a protein, growth factor, or cytokine includes all its isoforms functional fragments thereof, and analogues and mimetics thereof. Such reference also includes homologues from a variety of species, provided that the protein acts on the target cells in a similar fashion to the homologue from the same species as the target cells. For example, if human cells are being expanded, reference to bFGF would also include any non-human bFGF that acts on human cells, via the same receptor(s) as the human version. Those skilled in the art will appreciate that, even in the case of human cells, the aforementioned proteins need not be human proteins, since many non-human (e.g. animal) proteins are active on human cells. Similarly, the use of modified proteins that have similar activity to the native forms falls within the scope of the described methods and compositions. In other embodiments, isoforms of a given protein can be identified by those skilled in the art, for example by accessing the NCBI record, e.g. NCBI Gene ID: 2246 (accessed on Mar. 3, 2019), in the case of FGF1. FGF1α is a non-limiting example of an FGF1 isoform found in brain tissue.

In other embodiments, the growth factors comprise an FGF and TGF-beta. In still other embodiments, the growth factors comprise an FGF, TGF-beta, and PDGF. In more specific embodiments, the medium further comprises Transferrin, Insulin, or both Transferrin and Insulin. Alternatively or in addition, the growth factors further comprise oleic acid. In still other embodiments, the medium comprises Transferrin, Insulin, and Selenium.

In still other embodiments, the growth factors comprise an FGF and EGF. In still other embodiments, the medium further comprises Transferrin, Insulin, or both Transferrin and Insulin.

SFM and SPM

In general, the described SFM/SPM may be supplemented with factors intended to stimulate cell expansion in the absence of serum. Such medium is referred to herein as serum-replacement medium or SRM, and its use, for example in cell culture and expansion, is known in the art, and is described, for example, in Kinzebach et al. As used herein, “SRM” refers, in some embodiments, to entirely serum-free medium, and in other embodiments to serum-poor medium.

SRM formulations include MSC Nutristem® XF full medium (including the supplement) and MSC Nutristem® XF full medium (Biological Industries); Stempro® SFM and Stempro® SFM-XF (Thermo Fisher Scientific); PPRF-msc6; D-hESF10; TheraPEAK™ MSCGM-CDTM (Lonza, cat. no. 190632); and MesenCult-XF (Stem Cell Technologies, cat. no. 5429). The StemPro® media contain PDGF-BB, bFGF, and TGF-β, and Insulin (Chase et al). The composition of PPRF-msc6 is described in US 2010/0015710, which is incorporated herein by reference. D-hESF10 contains Insulin (10 mcg/mL); Transferrin (5 mcg/mL); oleic acid conjugated with bovine albumin (9.4 mcg/mL); FGF-2 (10 ng/mL); and TGF-β1 (5 ng/mL), as well as heparin (1 mg/mL) and standard medium components (Mimura et al).

In still other embodiments, a chemically-defined medium is utilized as the basal medium, to which growth factor(s) are added to obtain an SRM or expansion medium. Non-limiting examples of SRM and expansion media include a basal medium, supplemented with 5-50 ng/mL PDGF-BB, 1.5-15 ng/mL bFGF, and 0.2-2 ng/mL TGF-β; or with 50 ng/mL PDGF-BB, 15 ng/mL bFGF, and 2 ng/mL TGF-β; or with 5-20 ng/mL PDGF-BB; 1.5-5 ng/mL bFGF, and 0.2-0.8 ng/mL TGF-β; or with 5-10 ng/mL PDGF-BB, 1.5-4 ng/mL bFGF, and 0.2-0.4 ng/mL TGF-β; or with 5-10 ng/mL PDGF-BB, 1.5-3 ng/mL bFGF, and 0.2-0.3 ng/mL TGF-(3; or with about 5 ng/mL PDGF-BB, about 2 ng/mL bFGF, and about 0.2 ng/mL TGF-β. These media yielded similar results to Stempro® SFM-XF. Non-limiting examples of basal media are Nutristem® XF basal medium and DMEM/F-12, the latter being available commercially from Thermo Fisher Scientific (cat. no. 10565018). Alternatively, FGF-1 is used in place of bFGF. Those skilled in the art will be able to determine, in light of the information presented herein, concentrations of FGF-1 that have analogous effects of the indicated concentrations of bFGF, for example preferential expansion of particular populations of adherent stromal cells found in placenta.

Additional, non-limiting examples of SRM and expansion medium include a basal medium, supplemented with 30-300 mM HC, 2-30 ng/mL LIF, 0.1-0.5 ng/mL bFGF, and 0.02-0.08 ng/mL TGF-β; or with 50-200 mM HC, 5-20 ng/mL LIF, 0.2-0.5 ng/mL bFGF, and 0.02-0.06 ng/mL TGF-β; or with 70-150 mM HC, 7-15 ng/mL LIF, 0.3-0.5 ng/mL bFGF, and 0.03-0.06 ng/mL TGF-β; or with about 100 mM HC, about 10 ng/mL LIF, about 0.4 ng/mL bFGF, and about 0.04 ng/mL TGF-β. Alternatively, FGF-1 is used in place of bFGF. Those skilled in the art will be able to determine, in light of the information presented herein, concentrations of FGF-1 that have analogous effects of the indicated concentrations of bFGF, for example preferential expansion of particular populations of adherent stromal cells found in placenta. In other embodiments, a glucocorticoid is used in place of HC. Those skilled in the art will be able to determine, in light of the information presented herein, concentrations of a particular glucocorticoid that have analogous effects of the indicated concentrations HC, e.g. in facilitating differentiation-less expansion of placental cells.

Another SRM formulation is described in Rajaraman G et al and contains FGF-2 (10 ng/mL); epidermal growth factor (EGF) (10 ng/mL); 0.5% BSA; Insulin (10 mcg/mL); Transferrin (5.5 mcg/mL); 6.7 ng/mL sodium selenite, sodium pyruvate (11 mcg/mL); heparin (0.1 mg/mL); 10 nM linolenic acid.

Another SRM formulation for human stromal cells is described in U.S. Pat. No. 5,908,782 to Marshak and Holecek, incorporated herein by reference.

Other commercially available media include BD Mosaic™ hMSC serum-free medium (cat. no. 355701, BD Biosciences), CellGro™ (cat. no. 24803-0500, CellGenix, containing Insulin, albumin, and lecithin), HEScGRO (cat. no. SCM020, Merck Millipore), Mesenchymal stem cell growth medium DXF (cat. no. C-28019, PromoCell), MesenGro (cat. no. ZRD-MGro-500, StemRD), MSC Qualified PLUS™ (cat. no. PLS2, Compass Biomedical), MSC-Gro™ (SF, complete) (cat. no. SCO0B3, Vitro Biopharma), MSCGS-ACF (cat. no. 7572, ScienCell Research, mTeSR (cat. no. 5850, Stem Cell Technologies), PRIME-XV™ MSC Expansion SFM (cat. no. 31000, Irvine Scientific), RS-Novo™ and GEM-Novo (Kerry Bio-Sciences), MSCM-sf (ScienCell™), SPE-IV (cat. no. SPE-IV-EBM/500, Abecell-Bio), Stemline MSC expansion medium (cat. no. S1569, Sigma Aldrich), StemXVivo™ (cat. no. CCM014, R&D Systems, Inc), STK2 (Two Cells Co., Ltd.), and Ultrasor G (lyophilized) (cat. no. 15950-017, Pall Biosepra).

In certain embodiments, the described SRM comprises FGF (e.g. bFGF, also referred to as FGF-2), TGF-β (TGF-β, including all isotypes, for example TGFβ1, TGFβ2, and TGFβ3), or a combination thereof. In other embodiments, the SRM comprises FGF, TGF-β, and PDGF. In still other embodiments, the SRM comprises FGF and TGF-β, and lacks PDGF. Alternatively or in addition, Insulin is also present. In still other embodiments, an additional component selected from ascorbic acid, HC and fetuin is present; 2 components selected from ascorbic acid, HC and fetuin are present; or ascorbic acid, HC and fetuin are all present.

In certain embodiments of the described media, at least 2 of Transferrin, Insulin, Ascorbate are present. In other embodiments, all of Transferrin, Insulin, and Ascorbate are present. Optionally, L-glutamine is additionally present.

In still other embodiments, an anti-oxidant, e.g. selected from Ascorbate, Vitamin E (alpha-tocopherol), Lipoic acid, Dihydrolipoic acid, and Uric Acid, is present. In more specific embodiments, the anti-oxidant is present in addition to Transferrin and/or Insulin.

In other embodiments, the described SRM comprises FGF (e.g. bFGF), TGF-β, and Insulin. In additional embodiments, a component selected from Transferrin (5 mcg/mL) and oleic acid are present; or both Transferrin and oleic acid are present. Oleic acid can be, in some embodiments, conjugated with a protein, a non-limiting example of which is albumin. In some embodiments, the SRM comprises 5-20 ng/mL bFGF, 2-10 ng/mL TGF-β, and 5-20 ng/mL Insulin, or, in other embodiments, 7-15 ng/mL bFGF, 3-8 ng/mL TGF-β, and 7-15 ng/mL Insulin. In more specific embodiments, a component selected from 2-10 mcg/mL Transferrin and 5-20 mcg/mL oleic acid, or in other embodiments, a component selected from 3-8 mcg/mL Transferrin and 6-15 mcg/mL oleic acid, or in other embodiments the aforementioned amounts of both components (Transferrin and oleic acid) is/are also present.

In still other embodiments, the SRM further comprises a component, or in other embodiments 2, 3, or 4 components, selected from ethanolamine, glutathione, ascorbic acid, and albumin. Alternatively or in addition, the SRM further comprises a trace element, or in other embodiments, 2, 3, 4, or more than 4 trace elements. In some embodiments, the trace element(s) are selected from selenite, vanadium, copper, and manganese.

In still other embodiments, the described SRM comprises platelet lysate (van den Dolder et al), which serves, in more specific embodiments, as an activating component.

In yet other embodiments, the described SRM comprises bFGF and EGF. In more specific embodiments, the bFGF and EGF are present at concentrations independently selected from 5-40, 5-30, 5-25, 6-40, 6-30, 6-25, 7-40, 7-30, 7-25, 7-20, 8-, 8-17, 8-15, 8-13, 9-20, 9-17, 9-15, 10-15, 5-20, 5-10, 7-13, 8-12, 9-11, or 10 ng/mL. In certain embodiments, Insulin; and/or Transferrin is also present. In more specific embodiments, the Insulin and Transferrin are present at respective concentrations of 5-20 and 2-10; 6-18 and 3-8; or 8-15 and 4-7 mcg/mL. Alternatively or in addition, the SRM further comprises an additional component selected from BSA, selenite (e.g. sodium selenite), pyruvate (e.g. sodium pyruvate); heparin, and linolenic acid. In other embodiments 2 or more, or in other embodiments 3 or more, in other embodiments 4 or more, or in other embodiments all 5 of BSA, selenite, pyruvate, heparin, and linolenic acid are present. In more specific embodiments, the BSA, selenite, pyruvate, heparin, and linolenic acid are present at respective concentrations of 0.1-5%, 2-30 ng/mL, 5-25 mcg/mL, 0.05-0.2 mg/mL, and 5-20 nM; or in other embodiments at respective concentrations of 0.2-2%, 4-10 ng/mL, 7-17 mcg/mL, 0.07-0.15 mg/mL, and 7-15 nM; or in other embodiments the aforementioned amounts or 2 or more, or in other embodiments 3 or more, in other embodiments 4 or more, or in other embodiments all 5 of BSA, selenite, pyruvate, heparin, and linolenic acid are present.

In some embodiments, bFGF, when present, is present at a concentration of 1-10 nanograms per milliliter (ng/mL); or, in other embodiments, 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 8-13, 8-12, 9-11, 9-12, about 10, or 10 ng/mL. In other embodiments, Somatotropin is used instead, at one of the above concentrations.

In other embodiments, EGF, when present, is present at a concentration of 1-10 ng/mL; or, in other embodiments, 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 7-25, 7-22, 8-25, 8-22, 9-21, 10-20, 8-13, 8-12, 9-11, 9-12, about 10, or 10 ng/mL.

In other embodiments, TGF-β, when present, is present at a concentration of 1-10 ng/mL; or, in other embodiments, 1-25, 1-20, 1-15, 2-25, 3-25, 4-25, 5-25, 1-20, 1-15, 1-10, 1-8, 1-7, 1-6, 1-5, 2-20, 2-15, 2-10, 3-20, 3-15, 3-10, 3-8, 3-7, 4-8, 4-7, 4-6, 4.5-5.5, about 5, or 5 ng/mL. In other embodiments, WNT-3 is present instead of TGF, at one of the above concentrations.

In other embodiments, PDGF, when present, is present at a concentration of 1-10 ng/mL; or, in other embodiments, 1-50, 1-40, 1-30, 1-20, 1-15, 1-10, 1-8, 1-7, 1-6, 1-5, 1-4, 1-3, 1-2, 2-50, 2-40, 2-30, 2-20, 2-15, 2-10, 2-8, 2-7, 2-6, 2-5, 2-4, 3-50, 3-40, 3-30, 3-20, 3-15, 3-10, 3-8, 3-7, 3-6, 3-5, 3-4, 4-40, 4-30, 4-20, 5-40, 5-30, 5-20, 5-15, 5-12, 5-10, 10-20, 10-18, 10-16, or 10-15, 2-20, about 2, about 3, about 5, about 10, about 15, about 20, 2, 3, 5, 10, 15, or 20 ng/mL.

In other embodiments, HC, when present, is present at a concentration of 30-300 mM (millimolar); or, in other embodiments, 40-300, 50-300, 60-300, 70-300, 30-200, 40-200, 50-200, 60-200, 70-200, 30-150, 40-150, 50-150, 60-150, 70-150, 70-140, 70-130, 80-130, 80-120, 85-115, 90-110, 90-100, 100-110, 95-105, about 100, or 100 mM (millimolar).

In other embodiments, LIF, when present, is present at a concentration of 2-30 ng/mL; or, in other embodiments, 3-30, 4-30, 5-30, 6-30, 7-30, 2-20, 3-20, 4-20, 5-20, 6-20, 7-20, 2-15, 3-15, 4-15, 5-15, 6-15, 7-15, 7-14, 7-13, 8-15, 8-14, 8-13, 7-12, 8-12, 8.5-11.5, 9-11, 9-10, 10-11, 9.5-10.5, about 10, or 10 ng/mL. In other embodiments, IL-6 is used instead, at one of the above concentrations. In yet other embodiments, Somatotropin is used instead, at one of the above concentrations.

In other embodiments, Insulin, when present, is present at a concentration of 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 7-25, 7-22, 8-25, 8-22, 9-21, 10-20, 8-13, 8-12, 9-11, 9-12, about 10, or 10 micrograms per milliliter (mcg/mL).

In other embodiments, Transferrin, when present, is present at a concentration of 1-25, 2-25, 3-25, 4-25, 5-25, 1-20, 1-15, 1-10, 1-8, 1-7, 1-6, 1-5, 2-20, 2-15, 2-10, 3-20, 3-15, 3-10, 3-8, 3-7, 4-8, 4-7, 4-6, 4.5-5.5, about 5, or 5 mcg/mL.

In other embodiments, heparin, when present, is present at a concentration of 10-400, 10-300, 10-200, 20-400, 20-300, 20-200, 30-400, 30-300, 30-200, 30-150, 40-300, 40-200, 40-150, 50-300, 50-200, 50-150, 60-140, 70-140, 70-250, 70-220, 80-250, 80-220, 90-210, 100-200, 80-130, 80-120, 90-110, 90-120, about 100, or 100 ng/mL.

In other embodiments, linolenic acid, when present, is present at a concentration of 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 7-25, 7-22, 8-25, 8-22, 9-21, 10-20, 8-13, 8-12, 9-11, 9-12, about 10, or 10 nanomolar (nM).

In other embodiments, stem cell factor (SCF), when present, is present at a concentration of 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 7-25, 7-22, 8-25, 8-22, 9-21, 10-20, 8-13, 8-12, 9-11, 9-12, about 10, or 10 ng/mL.

In other embodiments, insulin-like growth factor-1 and/or 2 (IGF-1 and/or IGF-2) is present in the SRM. In more specific embodiments, IGF-1 and/or IGF-2 is present at a concentration of 10-250, 20-250, 30-250, 40-250, 50-250, 10-200, 10-150, 10-100, 10-80, 10-70, 10-60, 10-50, 20-200, 20-150, 20-100, 30-200, 30-150, 30-100, 30-80, 30-70, 40-80, 40-70, 40-60, 45-55, about 50, or 50 ng/mL.

In other embodiments, Keratinocyte Growth Factor (KGF) is present in the SRM. In more specific embodiments, KGF is present at a concentration of 5-100, 5-80, 5-60, 5-50, 5-40, 5-30, 5-20, 10-100, 10-80, 10-60, 10-50, 10-40, 10-30, 10-20, 20-100, 20-80, 20-60, 20-50, 20-40, 20-30, 15-25, 17-23, 18-22, 19-22, about 20, or 20 ng/mL.

In other embodiments, Interleukin 3 (IL-3) is present in the SRM. In more specific embodiments, IL-3 is present at a concentration of 0.5-10, 0.5-8, 0.5-6, 0.5-5, 0.5-4, 0.5-3, 0.5-2, 1-10, 1-8, 1-6, 1-5, 1-4, 1-3, 1-2, 2-10, 2-8, 2-6, 2-5, 2-4, 2-3, 1.5-2.5, 1.7-2.3, 1.8-2.2, 1.9-2.2, about 2, or 2 ng/mL.

In other embodiments, Interleukin 7 (IL-7) is present in the SRM. In more specific embodiments, IL-7 is present at a concentration of 0.5-10, 0.5-8, 0.5-6, 0.5-5, 0.5-4, 0.5-3, 0.5-2, 1-10, 1-8, 1-6, 1-5, 1-4, 1-3, 1-2, 2-10, 2-8, 2-6, 2-5, 2-4, 2-3, 1.5-2.5, 1.7-2.3, 1.8-2.2, 1.9-2.2, about 2, or 2 ng/mL.

Reference herein to the concentration of a cytokine or other factor indicates the concentration of the cytokine or factor in the medium when added to the cells, as opposed to the actual measured concentration of the cytokine or factor in the medium after addition to the cells, which maybe, in some embodiments, lower than its original concentration in the medium.

Those skilled in the art will appreciate, in light of the current disclosure, that various cytokines may be used as activating component(s). Non-limiting examples of cytokine-containing media are described, for example, in Carrero et al, Sullivan et al, Modrowski et al., and the references cited therein; and in US 2018/0037867 to Simmons et al., which is hereby incorporated by reference. In some embodiments, the cytokine is selected from one or more of VEGF, IL-1, SDF-1, TNF-alpha, and EPO. A non-limiting, exemplary formulation of an activating medium contains IL-1 and/or TNF-alpha. Those skilled in the art will appreciate that non-human versions of the described cytokines, as well as fragments, variations and mimetics thereof, may be used on human target cells, provided that they exhibit a significant portion of the activity of the human versions, when interacting with human cells. In certain embodiments, the concentration of IL-1 beta is 3-20 ng/mL (nanograms per milliliter), or in other embodiments 2-40, 2-35, 2-30, 2-25, 2-20, 2-15, 3-40, 3-35, 3-30, 3-25, 3-20, 3-15, 4-40, 4-35, 4-30, 4-25, 4-20, 4-15, 5-40, 5-35, 5-30, 5-25, 5-20, 5-15, 7-40, 7-35, 7-30, 7-25, 7-20, 7-15, 8-30, 8-25, 10-30, 10-25, 7-13, 8-12, 8.5-11.5, 9-11, about 10, or 10 ng/mL. Alternatively or additionally, the concentration of TNF-alpha is 3-20 ng/mL, or in other embodiments 2-40, 2-35, 2-30, 2-25, 2-20, 2-15, 3-40, 3-35, 3-30, 3-25, 3-20, 3-15, 4-40, 4-35, 4-30, 4-25, 4-20, 4-15, 5-40, 5-35, 5-30, 5-25, 5-20, 5-15, 7-40, 7-35, 7-30, 7-25, 7-20, 7-15, 8-30, 8-25, 10-30, 10-25, 7-13, 8-12, 8.5-11.5, 9-11, about 10, or 10 ng/mL. In more specific embodiments, TNF-alpha and IL-1-beta are both present a concentrations of 3-20 ng/mL, or in other embodiments, 2-40, 2-35, 2-30, 2-25, 2-20, 2-15, 3-40, 3-35, 3-30, 3-25, 3-20, 3-15, 4-40, 4-35, 4-30, 4-25, 4-20, 4-15, 5-40, 5-35, 5-30, 5-25, 5-20, 5-15, 7-40, 7-35, 7-30, 7-25, 7-20, 7-15, 8-30, 8-25, 10-30, 10-25, 7-13, 8-12, 8.5-11.5, 9-11, about 10, or 10 ng/mL. In certain embodiments, IL-1 alpha is used in place of IL-1 beta. The skilled person can readily determine, in light of the information presented herein, the concentrations of IL-1 alpha that will produce similar effects to the described concentrations of IL-1 beta. Alternatively or in addition, the activated ASC population is a maternal placental population. In other embodiments, the population is a fetal placental population. In other embodiments, the activation takes place on a 2D substrate. In other embodiments, the activation takes place in a bioreactor. In certain embodiments, the bioreactor contains a 3D substrate. Each possibility represents a separate embodiment.

IL-1 refers, in some embodiments, to a factor selected from IL-1α (interleukin-1 alpha (Uniprot Accession No. P01583; SEQ ID No. 40) and IL-1β interleukin-1 beta (Uniprot Accession No. P01584; SEQ ID No. 41). IL-1 proteins and their functions are known in the art, and are described, inter alia, in Malik and Kanneganti and the references cited therein. Those skilled in the art will appreciate that non-human versions of IL-1 proteins, as well as fragments, variations and mimetics thereof, may be used on human target cells, provided that they exhibit a significant portion of the activity of the human versions, when interacting with human cells. Relevant IL-1 activities include stimulation of IL-1R1 (Interleukin-1 receptor type 1; Uniprot Accession No. P14778; SEQ ID No. 42).

TNF-alpha refers to tumor necrosis factor alpha (Uniprot Accession No. P01375; SEQ ID No. 43). TNF-alpha proteins and their functions are known in the art, and are described, inter alia, in Aggarwal B B et al and the references cited therein. Those skilled in the art will appreciate that non-human versions of TNF-alpha proteins, as well as fragments, variations and mimetics thereof, may be used on human target cells, provided that they exhibit a significant portion of the activity of the human versions, when interacting with human cells. Relevant TNF-alpha activities include binding of Tumor necrosis factor receptor superfamily member 1A (Uniprot Accession No. P19438; SEQ ID No. 44).

In other embodiments, the activating medium contains VEGF. VEGF refers, in some embodiments, to Vascular endothelial growth factor, e.g. isoforms A, B, C, or D (Uniprot Accession Nos. P15692, P49765, P49767, 043915; SEQ ID Nos. 45-48). In other embodiments, the VEGF protein is Placenta growth factor (Uniprot Accession No. P49763; SEQ ID No. 49). VEGF proteins and their functions are known in the art. Those skilled in the art will appreciate that non-human versions of VEGF proteins, as well as fragments, variations and mimetics thereof, may be used on human target cells, provided that they exhibit a significant portion of the activity of the human versions, when interacting with human cells. Relevant VEGF activities include stimulation of Vascular endothelial growth factor receptor 2 (Uniprot Accession No. P35968; SEQ ID No. 50). In some embodiments, VEGF is the only activating component; while in other embodiments, VEGF is used together with another activating component, e.g. an activating component mentioned herein. Alternatively or in addition, VEGF is used at a concentration of 1-20 ng/mL; or in other embodiments 1-10 ng/mL; or in other embodiments 1-20, 1-15, 1-12, 2-20, 2-15, 2-12, 3-20, 3-15, 3-12, 5-20, 5-15, 5-12, 1-8, 1-6, 1-5, 1-4, 1-3, 1-2, 2-10, 2-8, 2-6, 2-5, 2-4, 2-3, 3-10, 3-8, 3-6, 3-5, 3-4, 4-10, 4-8, 4-6, 4-5, 5-10, 5-8, 6-10, 6-8, or 8-10 ng/mL. Alternatively or in addition, the activated ASC population is a maternal placental population. In other embodiments, the population is a fetal placental population. In other embodiments, the activation takes place on a 2D substrate. In other embodiments, the activation takes place in a bioreactor. In certain embodiments, the bioreactor contains a 3D substrate. Each possibility represents a separate embodiment.

In other embodiments, the activating medium contains SDF-1. SDF-1 refers, in some embodiments, to Stromal cell-derived factor 1 (Uniprot Accession No. P48061; SEQ ID No. 51). SDF-1 proteins and their functions are known in the art. Those skilled in the art will appreciate that non-human versions of SDF-1 proteins, as well as fragments, variations and mimetics thereof, may be used on human target cells, provided that they exhibit a significant portion of the activity of the human versions, when interacting with human cells. Relevant SDF-1 activities include stimulation of C-X-C chemokine receptor type 4 (CXCR4; Uniprot Accession No. P61073; SEQ ID No. 52). In some embodiments, SDF-1 is the only activating component; while in other embodiments, SDF-1 is used together with another activating component, e.g. an activating component mentioned herein. Alternatively or in addition, SDF-1 is used at a concentration of 1-20 ng/mL; or in other embodiments 1-10 ng/mL; or in other embodiments 1-20, 1-15, 1-12, 2-20, 2-15, 2-12, 3-20, 3-15, 3-12, 5-20, 5-15, 5-12, 1-8, 1-6, 1-5, 1-4, 1-3, 1-2, 2-10, 2-8, 2-6, 2-5, 2-4, 2-3, 3-10, 3-8, 3-6, 3-5, 3-4, 4-10, 4-8, 4-6, 4-5, 5-10, 5-8, 6-10, 6-8, or 8-10 ng/mL. Alternatively or in addition, the activated ASC population is a maternal placental population. In other embodiments, the population is a fetal placental population. In other embodiments, the activation takes place on a 2D substrate. In other embodiments, the activation takes place in a bioreactor. In certain embodiments, the bioreactor contains a 3D substrate. Each possibility represents a separate embodiment.

In other embodiments, the activating medium contains EPO. EPO refers, in some embodiments, to Erythropoietin (Uniprot Accession No. P01588; SEQ ID No. 53). EPO proteins and their functions are known in the art. Those skilled in the art will appreciate that non-human versions of EPO proteins, as well as fragments, variations and mimetics thereof, may be used on human target cells, provided that they exhibit a significant portion of the activity of the human versions, when interacting with human cells. Relevant EPO activities include stimulation of Erythropoietin receptor (Uniprot Accession No. P19235; SEQ ID No. 54). In some embodiments, EPO is the only activating component; while in other embodiments, EPO is used together with another activating component, e.g. an activating component mentioned herein. Alternatively or in addition, EPO is used at a concentration of 1-20 ng/mL; or in other embodiments 1-10 ng/mL; or in other embodiments 1-20, 1-15, 1-12, 2-20, 2-15, 2-12, 3-20, 3-15, 3-12, 5-20, 5-15, 5-12, 1-8, 1-6, 1-5, 1-4, 1-3, 1-2, 2-10, 2-8, 2-6, 2-5, 2-4, 2-3, 3-10, 3-8, 3-6, 3-5, 3-4, 4-10, 4-8, 4-6, 4-5, 5-10, 5-8, 6-10, 6-8, or 8-10 ng/mL. Alternatively or in addition, the activated ASC population is a maternal placental population. In other embodiments, the population is a fetal placental population. In other embodiments, the activation takes place on a 2D substrate. In other embodiments, the activation takes place in a bioreactor. In certain embodiments, the bioreactor contains a 3D substrate. Each possibility represents a separate embodiment.

In yet other embodiments, the activation factors comprise a factor selected from IL-4 (interleukin-4; Uniprot Accession No. P05112; SEQ ID No. 55) and IL-13 (interleukin 13; Uniprot Accession No. P35225; SEQ ID No. 82). IL-4 and IL-13 proteins and their functions are known in the art, and are described, inter alia, in Lin and Leonard, Bian Z M et al, and the references cited therein. Those skilled in the art will appreciate that non-human versions of IL-4 and IL-13 proteins, as well as fragments, variations and mimetics thereof, may be used on human target cells, provided that they exhibit a significant portion of the activity of the human versions, when interacting with human cells. Relevant IL-4 and IL-13 activities include binding of Interleukin-4 receptor (Uniprot Accession Nos. P24394 and P31785; SEQ ID Nos. 56-57). In some embodiments, IL-4 or IL-13 is used at a concentration of 1-10 ng/mL, or in other embodiments 1-8, 1-6, 1-5, 1-4, 1-3, 1-2, 2-10, 2-8, 2-6, 2-5, 2-4, 2-3, 3-10, 3-8, 3-6, 3-5, 3-4, 4-10, 4-8, 4-6, 4-5, 5-10, 5-8, 6-10, 6-8, or 8-10 ng/mL. In certain embodiments, IL-4 or IL-13 is included in addition of IL-1-beta and TNF-alpha.

In still other embodiments, there is provided a method of expanding an ASC population (which may be, in some embodiments, a placental cell population), comprising incubating the ASC population in a chemically-defined basal medium with the addition of growth factors, which may be, in certain embodiments, an expansion medium or SRM described herein. In other embodiments, there is a provided a method of expanding placental cells to obtain a primarily, or entirely, maternal population, comprising incubation with the described media. The incubation is followed, in some embodiments, by incubation in a second expansion medium or SRM, wherein the second medium comprises one or more activating components. Other than the activating components, the second expansion medium or SRM may be the same or different from the first expansion medium or SRM.

In other embodiments, the growth factors comprise 2 or more of FGF (e.g. bFGF), TGF-beta, LIF, and a glucocorticoid. In other embodiments, the growth factors comprise 3 or more of FGF, TGF-beta, LIF, and a glucocorticoid. In still other embodiments, the factors comprise FGF, TGF-beta, LIF, and a glucocorticoid. In other embodiments, the factors comprise, or in other embodiments consist of, FGF, TGF-beta, and a glucocorticoid. In yet other embodiments, the expansion medium used in the described methods consists of, or in other embodiments consists essentially of, a basal medium, with the addition of FGF, TGF-beta, LIF, and a glucocorticoid; one or more fatty acids; Insulin; Transferrin; optionally one or more other carrier proteins; Selenium; one or more antioxidants; and one or more pH buffers. In still other embodiments, L-Glutamine is also present. In other embodiments, EGF is used in place of FGF, e.g. at a concentration of 0.1-1 ng/mL; or, in other embodiments, 0.2-1, 0.3-1, 0.4-1, 0.5-1, 0.2-2, 0.2-1.5, 0.2-5, 0.3-2, 0.3-1.5, 0.3-1, 0.2-0.8, 0.3-0.7, or 0.4-0.6 ng/mL. In still other embodiments, EGF is used in addition to FGF.

In other embodiments, the expansion medium comprises, or in other embodiments consists of, a basal medium, supplemented with one or more fatty acids; Insulin; Transferrin; optionally one or more other carrier proteins; Selenium; one or more antioxidants; and one or more pH buffers; to which is added: 30-300 mM HC, 2-30 ng/mL LIF, 0.1-0.5 ng/mL bFGF, and 0.02-0.08 ng/mL TGF-β; or 50-200 mM HC, 5-20 ng/mL LIF, 0.2-0.5 ng/mL bFGF, and 0.02-0.06 ng/mL TGF-β; or 70-150 mM HC, 7-15 ng/mL LIF, 0.3-0.5 ng/mL bFGF, and 0.03-0.06 ng/mL TGF-β; or about 100 mM HC, about 10 ng/mL LIF, about 0.4 ng/mL bFGF, and about 0.04 ng/mL TGF-β. In other embodiments, another glucocorticoid, a non-limiting example of which is dexamethasone, is used in place of HC. In still other embodiments, L-Glutamine is also present.

In certain embodiments, the ASC population is incubated in the described first expansion medium for at least 17 doublings, or in other embodiments at least 12, at least 15, at least 18, 12-30, 12-25, 15-30, 15-25, 16-25, 17-25, or 18-25 doublings. In other embodiments, the incubation is carried out for a defined number of passages, for example 2-3, or in other embodiments 1-4, 1-3, 1-2, or 2-4; or a defined number of population doublings, for example 4-7, or in other embodiments at least 4, at least 5, at least 6, at least 7, at least 8, 4-10, 4-9, 4-8, 5-10, 5-9, or 5-8. The cells are then cryopreserved, then subjected to additional culturing, e.g. in the same medium. In some embodiments, the additional culturing is performed for 6-10 population doublings, or in other embodiments at least 6, at least 7, at least 8, at least 9, at least 10, 6-20, 7-20, 8-20, 9-20, 10-20, 6-15, 7-15, 8-15, 9-15, or 10-15 population doublings. Alternatively, the additional culturing is performed for 2-3 passages, or in other embodiments at least 1, at least 2, at least 3, 1-5, 1-4, 1-3, 2-5, or 2-4 passages.

In other embodiments, the ASC population is incubated for a defined number of days, for example 4-10, 5-10, 6-10, 4-9, 4-8, 4-7, 5-9, 5-8, 5-7, 6-10, 6-9, or 6-8; or a defined number of population doublings, for example at least 3, at least 4, at least 5, at least 6, 3-10, 3-9, 3-8, 4-10, 4-9, or 4-8. The cells are then subjected to additional culturing in a bioreactor, e.g. in the same medium. In some embodiments, the bioreactor culturing is performed for at least 4, at least 5, at least 6, at least 7, at least 8, at least 9, 4-10, 4-9, 4-8, 5-10, 5-9, 5-8, 6-10, 6-9, or 6-8 population doublings; or, in other embodiments, for at least 4, at least 5, at least 6, at least 7, 4-15, 4-12, 4-10, 4-9, 4-8, 4-7, 4-15, 5-12, 5-10, 5-9, 5-8, 5-7, 6-15, 6-12, 6-10, 6-9, 6-8, or 6-7 days. In certain embodiments, the bioreactor contains 3D carriers, on which the cells are cultured.

In still other embodiments, there is provided a method of expanding an ASC population (which may be, in some embodiments, a placental cell population), comprising incubating the ASC population in a first expansion medium; followed by incubation in a second expansion medium, wherein the second expansion medium further comprises one or more activating components. Other than the activating components, the second expansion medium may be the same or different from the first expansion medium. In other embodiments, there is a provided a method of expanding placental cells to obtain a primarily, or entirely, fetal population, comprising incubation with the described media.

In certain embodiments, the growth factors comprise 2 or more of FGF (e.g. bFGF) (a.k.a. Fibroblast Growth Factor 2; Uniprot accession no. P09038; SEQ ID No. 58), TGF-beta, and PDGF (e.g. PDGF-BB). In still other embodiments, the factors comprise, or in other embodiments consist of, bFGF, TGF-beta, and PDGF (e.g. PDGF-BB). In yet other embodiments, the expansion medium used in the described methods consists of, or in other embodiments consists essentially of, a basal medium, with the addition of bFGF, TGF-beta, and PDGF (e.g. PDGF-BB); one or more fatty acids; Insulin; Transferrin; optionally one or more other carrier proteins; Selenium; one or more antioxidants; and one or more pH buffers. In still other embodiments, L-Glutamine is also present.

In other embodiments, the expansion medium comprises, or in other embodiments consists of, a basal medium, supplemented with one or more fatty acids; Insulin; Transferrin; optionally one or more other carrier proteins; Selenium; one or more antioxidants; one or more pH buffers; and optionally L-Glutamine; to which is added 5-50 ng/mL PDGF-BB, 1.5-15 ng/mL bFGF, and 0.2-2 ng/mL TGF-β; or 50 ng/mL PDGF-BB, 15 ng/mL bFGF, and 2 ng/mL TGF-β; or 5-20 ng/mL PDGF-BB; 1.5-5 ng/mL bFGF, and 0.2-0.8 ng/mL TGF-β; or 5-10 ng/mL PDGF-BB, 1.5-4 ng/mL bFGF, and 0.2-0.4 ng/mL TGF-β; or 5-10 ng/mL PDGF-BB, 1.5-3 ng/mL bFGF, and 0.2-0.3 ng/mL TGF-β; or about 5 ng/mL PDGF-BB, about 2 ng/mL bFGF, and about 0.2 ng/mL TGF-β.

In certain embodiments, a PL, a non-limiting example of which is human PL, is used as an activating component in the described methods and compositions.

As mentioned, the described methods are optionally preceded by an earlier step wherein cells are cultured in a medium containing over 5% animal serum. The serum-containing medium can be, in certain embodiments, any standard growth medium. Non-limiting examples, for exemplary purposes only, are DMEM+10% FBS and DMEM+5% human serum. As described herein, a non-limiting example of these embodiments is use of standard growth medium to incubate and expand ASC, as the next step following their extraction from the source tissue, followed by expansion in SFM or SPM, which is optionally, in turn followed by expansion in a medium comprising activating component(s), or a medium containing over 5% animal serum. In certain embodiments, the initial use of serum-containing medium, in some scenarios, facilitates initial attachment and expansion of cells after their extraction. In other embodiments, a platelet lysate (PL), a non-limiting example of which is human PL, is used in place of serum in the initial incubation. In other embodiments, ASC are expanded from the outset in SPM or SFM.

In certain embodiments, the aforementioned optional step of incubating the ASC population in a serum-containing medium immediately after extraction is performed for a single passage, or in other embodiments for 1-3 passages, or 1-2 passages. In other embodiments, the optional step is performed for 2-5 population doublings, or in other embodiments for 2-20, 2-15, 2-10, 2-8, 2-6, or 2-5 doublings. Those skilled in the art will appreciate that it may be difficult to determine an exact population doubling level (PDL) between extraction of cells from tissue and the first passage. In such case, if necessary the population doublings at this first stage may be estimated. Typical population doubling values prior to the first passage are below 5, often ranging from 2-5.

An exemplary, non-limiting extraction protocol from placenta into serum-containing medium is described in Example 1 of International Patent Application WO 2016/098061, in the name of Esther Lukasiewicz Hagai et al, published on Jun. 23, 2016, which is incorporated herein by reference in its entirety. Following initial extractions, cells are, in further embodiments, expanded in SFM or SRM, in some embodiments for about 2-3 passages, or typically about 4-12 population doublings after the first passage. In yet further embodiments, the culturing is optionally followed by cell concentration, formulation, and cryopreservation, and the optional thawing and additional culturing. In certain embodiments, the initial culturing is all carried out on a 2D substrate. Those skilled in the art will appreciate that non-limiting examples of cryopreservation excipients include DMSO and serum. Other embodiments of cryopreservation media are described herein.

In certain embodiments, the aforementioned culturing steps are followed by culturing in a bioreactor, which is, in some embodiments, performed in SFM, or in other embodiments, SRM. In other embodiments, the bioreactor medium contains activating component(s), or in still other embodiments, serum. In more particular embodiments, the bioreactor culture is performed for 2-5 additional doublings, or in other embodiments up to 10 additional doublings. In certain embodiments, the bioreactor contains a 3D substrate. In other embodiments, a PL, a non-limiting example of which is human PL, is used as an activating component. In still other embodiments, one or more cytokines are used as activating component(s). Optionally, bioreactor growth may be followed by any or all of harvest, cell concentration, washing, formulation, and/or cryopreservation.

In some embodiments, the step of incubating the ASC population in a first medium is performed on a 2D substrate; and at least a portion of the subsequent step (incubating the first expanded cell population in a second medium) is performed on a 3D substrate. In certain embodiments, the 3D substrate is in a bioreactor. Alternatively or in addition, the 3D substrate is a synthetic adherent material. In still other embodiments, the aforementioned subsequent step is initiated on a 2D substrate for a duration of 2-6, or in other embodiments at least 2, at least 3, at least 4, at least 5, at least 6, 1-5, 2-5, 3-5, 1-2, 1-3, or 1-5 cell doublings, before performing additional expansion in a second medium on a 3D substrate. The 2D substrate on which the subsequent step is initiated may be the same or different from the 2D substrate on which the step of incubating the ASC population in a first medium is performed.

In other embodiments, the step of incubating the ASC population in a first medium is performed in a batch culture, and at least a portion of the subsequent step is performed under perfusion. In still other embodiments, the aforementioned subsequent step is initiated in a batch culture for a duration of 2-6, or in other embodiments at least 2, at least 3, at least 4, at least 5, at least 6, 1-5, 2-5, 3-5, 1-2, 1-3, or 1-5-cell doublings, before performing additional expansion in a second medium under perfusion.

In certain embodiments, cells are extracted and/or subject to 2D culturing, cryopreservation, 3D culturing, harvesting, and formulation, for example as described in WO 2017/212309 to Eytan Abraham et al, which is incorporated by reference herein.

Adherent Materials

In various embodiments, “an adherent material” refers to a material that is synthetic, or in other embodiments naturally occurring, or in other embodiments a combination thereof. In certain embodiments, the material is non-cytotoxic (or, in other embodiments, is biologically compatible). Alternatively or in addition, the material is fibrous, which may be, in more specific embodiments, a woven fibrous matrix, a non-woven fibrous matrix, or either. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a polycarbonate, a cellulose acetate, a glass fiber, a ceramic particle, a poly-L-lactic acid, and an inert metal fiber. Other embodiments include Matrigel™, an extra-cellular matrix component (e.g., Fibronectin, Chondronectin, Laminin; or a fragment thereof), and a collagen. In more particular embodiments, the material may be selected from a polyester and a polypropylene. In various embodiments, an “adherent material” refers to a material that is synthetic, or in other embodiments naturally occurring, or in other embodiments a combination thereof. In certain embodiments, the material is non-cytotoxic (or, in other embodiments, is biologically compatible). Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polycarbonates, polyfluorochloroethylenes, polyvinyl chlorides, polystyrenes, polysulfones, cellulose acetates, and poly-L-lactic acids. Other embodiments include Matrigel™, an extra-cellular matrix component (e.g., Fibronectin, Chondronectin, Laminin), and a collagen.

Alternatively or in addition, the adherent material is fibrous, which may be, in more specific embodiments, a woven fibrous matrix, a non-woven fibrous matrix, or either. In still other embodiments, the material exhibits a chemical structure such as charged surface groups, which allows cell adhesion, e.g. polyesters, polypropylenes, polyalkylenes, polyfluorochloroethylenes, polyvinyl chlorides, polystyrenes, polysulfones, cellulose acetates, and poly-L-lactic acids. In more particular embodiments, the material may be selected from a polyester and a polypropylene.

Bioreactors

In certain embodiments, the described methods, or certain steps thereof, are performed in a bioreactor. In some embodiments, the bioreactor comprises a container for holding medium and a 3D attachment (carrier) substrate disposed therein, and a control apparatus, for controlling pH, temperature, and oxygen levels and optionally other parameters. In more specific embodiments, the 3D substrate is in a packed bed configuration. Alternatively or in addition, the bioreactor contains ports for the inflow and outflow of fresh medium and gases.

In certain embodiments, the aforementioned bioreactor is a packed-bed bioreactor. In some embodiments, the bioreactor comprises a container for holding medium, and a control apparatus, for controlling pH, temperature, and oxygen levels and optionally other parameters. In more specific embodiments, the bioreactor also contains a 3D substrate. Alternatively or in addition, the bioreactor contains ports for the inflow and outflow of fresh medium and gases.

In certain embodiments, the bioreactor is connected to an external medium reservoir (e.g. that is used to perfuse the bioreactor).

The term packed-bed bioreactor, except where indicated otherwise, refers to a bioreactor in which the cellular growth substrate is not ordinarily lifted from the bottom of the incubation vessel in the presence of growth medium. For example, the substrate may have sufficient density to prevent being lifted and/or it may be packed by mechanical pressure to present it from being lifted. The substrate may be either a single body or multiple bodies. Typically, the substrate remains substantially in place during perfusion at the standard perfusion rate of the bioreactor. In certain embodiments, the definition does not exclude that the substrate may be lifted at unusually fast perfusion rates, for example greater than 200 rpm.

Examples of bioreactors include, but are not limited to, a continuous stirred tank bioreactor, a CelliGen® bioreactor system (New Brunswick Scientific (NBS) and a BIOFLO 310 bioreactor system (New Brunswick Scientific (NBS).

In certain embodiments, a bioreactor is capable, in certain embodiments, of expansion of cells on a 3D substrate under controlled conditions (e.g. pH, temperature and oxygen levels) and with growth medium perfusion, which in some embodiments is constant perfusion and in other embodiments is adjusted in order to maintain target levels of glucose or other components. Furthermore, the cell cultures can be directly monitored for concentrations of glucose, lactate, glutamine, glutamate and ammonium. The glucose consumption rate and the lactate formation rate of the adherent cells enable, in some embodiments, measurement of cell growth rate and determination of the harvest time.

In some embodiments, a continuous stirred tank bioreactor is used, where a culture medium is continuously fed into the bioreactor and a product is continuously drawn out, to maintain a time-constant steady state within the reactor. A stirred tank bioreactor with a fibrous bed basket is available for example from New Brunswick Scientific Co., Edison, N.J.). Additional bioreactors that may be used, in some embodiments, are stationary-bed bioreactors; and air-lift bioreactors, where air is typically fed into the bottom of a central draught tube flowing up while forming bubbles, and disengaging exhaust gas at the top of the column. Additional possibilities are cell-seeding perfusion bioreactors with polyactive foams [as described in Wendt, D. et al., Biotechnol Bioeng 84: 205-214, (2003)] and radial-flow perfusion bioreactors containing tubular poly-L-lactic acid (PLLA) porous scaffolds [as described in Kitagawa et al., Biotechnology and Bioengineering 93(5): 947-954 (2006). Other bioreactors which can be used are described in U.S. Pat. Nos. 6,277,151; 6,197,575; 6,139,578; 6,132,463; 5,902,741; and 5,629,186, which are incorporated herein by reference.

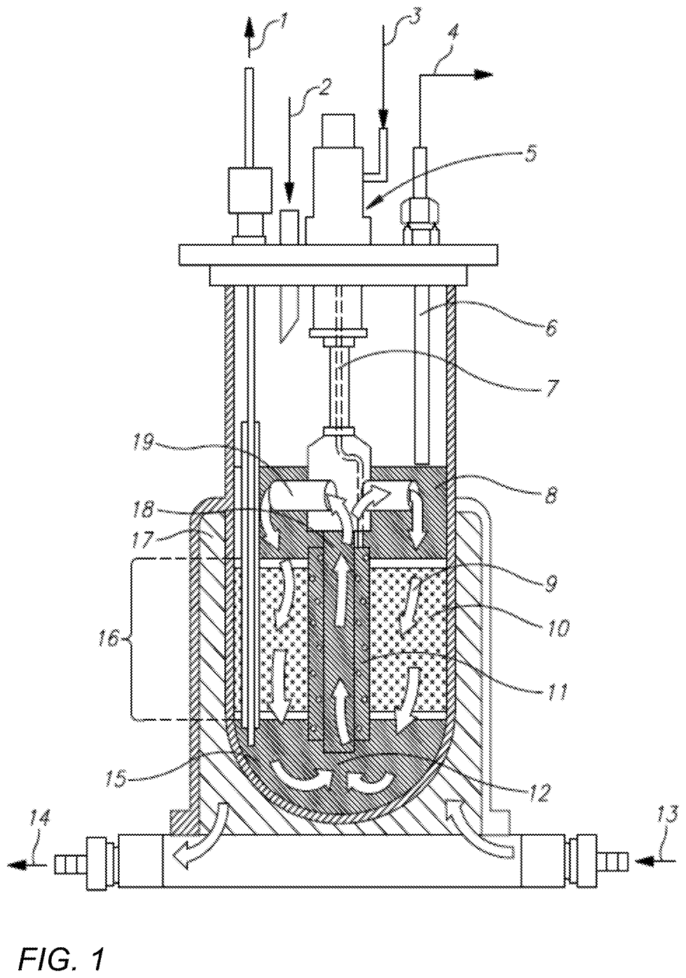

Another exemplary bioreactor, the CelliGen 310 Bioreactor, is depicted in FIG. 1. In the depicted embodiment, A Fibrous-Bed Basket (16) is loaded with polyester disks (10). In some embodiments, the vessel is filled with deionized water or isotonic buffer via an external port (1 [this port may also be used, in other embodiments, for cell harvesting]) and then optionally autoclaved. In other embodiments, following sterilization, the liquid is replaced with growth medium, which saturates the disk bed as depicted in (9). In still further embodiments, temperature, pH, dissolved oxygen concentration, etc., are set prior to inoculation. In yet further embodiments, a slow initial stirring rate is used to promote cell attachment, then the stirring rate is increased. Alternatively or addition, perfusion is initiated by adding fresh medium via an external port (2). If desired, metabolic products may be harvested from the cell-free medium above the basket (8). In some embodiments, rotation of the impeller creates negative pressure in the draft-tube (18), which pulls cell-free effluent from a reservoir (15) through the draft tube, then through an impeller port (19), thus causing medium to circulate (12) uniformly in a continuous loop. In still further embodiments, adjustment of a tube (6) controls the liquid level; an external opening (4) of this tube is used in some embodiments for harvesting. In other embodiments, a ring sparger (not visible), is located inside the impeller aeration chamber (11), for oxygenating the medium flowing through the impeller, via gases added from an external port (3), which may be kept inside a housing (5), and a sparger line (7). Alternatively or in addition, sparged gas confined to the remote chamber is absorbed by the nutrient medium, which washes over the immobilized cells. In still other embodiments, a water jacket (17) is present, with ports for moving the jacket water in (13) and out (14).

In certain embodiments, a perfused bioreactor is used, wherein the perfusion chamber contains carriers. The carriers may be, in more specific embodiments, selected from macrocarriers, microcarriers, or either. Non-limiting examples of microcarriers that are available commercially include alginate-based (GEM, Global Cell Solutions), dextran-based (Cytodex®, GE Healthcare), collagen-based (Cultispher®, Percell Biolytica), and polystyrene-based (SoloHill Engineering) microcarriers. In certain embodiments, the microcarriers are packed inside the perfused bioreactor.

In some embodiments, the carriers in the perfused bioreactor are packed, for example forming a packed bed, which is submerged in a nutrient medium. Alternatively or in addition, the carriers may comprise an adherent material. In other embodiments, the surface of the carriers comprises an adherent material, or the surface of the carriers is adherent. In still other embodiments, the material exhibits a chemical structure such as charged surface exposed groups, which allows cell adhesion.

Alternatively or in addition, the carriers comprise a fibrous material, optionally an adherent, fibrous material, which may be, in more specific embodiments, a woven fibrous matrix, a non-woven fibrous matrix, or either. Non-limiting examples of fibrous carriers are New Brunswick Scientific Fibracel® carriers, available commercially from of Eppendorf AG, Germany, and made of polyester and polypropylene; and BioNOC II carriers, available commercially from CESCO BioProducts (Atlanta, Ga.) and made of PET (polyethylene terephthalate). In certain embodiments, the referred-to fibrous matrix comprises a polyester, a polypropylene, a polyalkylene, a polyfluorochloroethylene, a polyvinyl chloride, a polystyrene, or a polysulfone. In more particular embodiments, the fibrous matrix is selected from a polyester and a polypropylene.

In other embodiments, cells are produced using a packed-bed spinner flask. In more specific embodiments, the packed bed may comprise a spinner flask and a magnetic stirrer. The spinner flask may be fitted, in some embodiments, with a packed bed apparatus, which may be, in more specific embodiments, a fibrous matrix; a non-woven fibrous matrix; non-woven fibrous matrix comprising polyester; or a non-woven fibrous matrix comprising at least about 50% polyester. In more specific embodiments, the matrix may be similar to the CelliGen™ Plug Flow bioreactor which is, in certain embodiments, packed with Fibra-Cel® (or, in other embodiments, other carriers). The spinner is, in certain embodiments, batch fed (or in other alternative embodiments fed by perfusion), fitted with one or more sterilizing filters, and placed in a tissue culture incubator. In further embodiments, cells are seeded onto the scaffold by suspending them in medium and introducing the medium to the apparatus. In still further embodiments, the stirring speed is gradually increased, for example by starting at 40 RPM for 4 hours, then gradually increasing the speed to 120 RPM. In certain embodiments, the glucose level of the medium may be tested periodically (i.e. daily), and the perfusion speed adjusted maintain an acceptable glucose concentration, which is, in certain embodiments, between 400-700 mg\liter, between 450-650 mg\liter, between 475-625 mg\liter, between 500-600 mg\liter, or between 525-575 mg\liter. In yet other embodiments, at the end of the culture process, the carriers are removed from the packed bed and, in some embodiments, washed with isotonic buffer, and the cells are processed or removed from the carriers by agitation and/or enzymatic digestion.

In certain embodiments, the bioreactor is seeded at a concentration of between 10,000-2,000,000 cells/mL of medium, or, in various embodiments 20,000-2,000,000, 30,000-1,500,000, 40,000-1,400,000, 50,000-1,300,000, 60,000-1,200,000, 70,000-1,100,000, 80,000-1,000,000, 80,000-900,000, 80,000-800,000, 80,000-700,000, 80,000-600,000, 80,000-500,000, 80,000-400,000, 90,000-300,000, 90,000-250,000, 90,000-200,000, 100,000-200,000, 110,000-1,900,000, 120,000-1,800,000, 130,000-1,700,000, or 140,000-1,600,000 cells/mL.

In still other embodiments, between 1-20×10⁶ cells per gram (gr) of carrier (substrate) are seeded, or in other embodiments 1.5-20×10⁶ cells/gr carrier, or in other embodiments 1.5-18×10⁶ cells/gr carrier, or in other embodiments 1.8-18×10⁶ cells/gr carrier, or in other embodiments 2-18×10⁶ cells/gr carrier, or in other embodiments 3-18×10⁶ cells/gr carrier, or in other embodiments 2.5-15×10⁶ cells/gr carrier, or in other embodiments 3-15×10⁶ cells/gr carrier, or in other embodiments 3-14×10⁶ cells/gr carrier, or in other embodiments 3-12×10⁶ cells/gr carrier, or in other embodiments 3.5-12×10⁶ cells/gr carrier, or in other embodiments 3-10×10⁶ cells/gr carrier, or in other embodiments 3-9×10⁶ cells/gr carrier, or in other embodiments 4-9×10⁶ cells/gr carrier, or in other embodiments 4-8×10⁶ cells/gr carrier, or in other embodiments 4-7×10⁶ cells/gr carrier, or in other embodiments 4.5-6.5×10⁶ cells/gr carrier.

Adherent Stromal Cells

In certain embodiments, the cells that are subjected to the described methods are placenta-derived adherent cells, which may be, in more specific embodiments, adherent stromal cells. Except where indicated otherwise herein, the terms “placenta”, “placental tissue”, and the like refer to any portion of the placenta. Placenta-derived adherent cells may be obtained, in various embodiments, from either fetal or, in other embodiments, maternal regions of the placenta, or in other embodiments, from both regions; or the cells may be substantially entirely fetal cells, or maternal cells; enriched for fetal cells, or maternal cells; or predominantly fetal cells, or predominantly maternal cells. More specific embodiments of maternal sources are the decidua basalis and the decidua parietalis. More specific embodiments of fetal sources are the amnion, the chorion, and the villi. In particular embodiments, the amnion and chorion are removed, and most or all of the remaining placental tissue is subjected to enzymatic treatment and physical disruption.

In certain embodiments, tissue specimens are washed in a physiological buffer [e.g., phosphate-buffered saline (PBS) or Hank's buffer]. Single-cell suspensions can be made, in other embodiments, by treating the tissue with a digestive enzyme (see below) or/and physical disruption, a non-limiting example of which is mincing and flushing the tissue parts through a nylon filter or by gentle pipetting (Falcon, Becton, Dickinson, San Jose, Calif.) with washing medium. In some embodiments, the tissue treatment includes use of a DNAse, a non-limiting example of which is Benzonase from Merck.

In other embodiments, at least 50%, at least 60%, at least 70%, at least 75%, at least 80%, at least 85%, at least 90%, at least 92%, at least 95%, at least 96%, at least 97%, at least 98%, at least 99%, at least 99.5%, at least 99.7%, or at least 99.9% of the cells that are expanded are maternally-derived cells.

In yet other embodiments, at least 50%, at least 60%, at least 70%, at least 75%, at least 80%, at least 85%, at least 90%, at least 92%, at least 95%, at least 96%, at least 97%, at least 98%, at least 99%, at least 99.5%, at least 99.7%, or at least 99.9% of the described cells are fetal cells. As provided herein, fetal cells can be successfully activated with cytokines, following expansion in serum-free medium.

Placental cells may be obtained, in various embodiments, from a full-term or pre-term placenta. “Full-term” placenta in this regard refers to a placenta whose gestational age is at least 36 weeks. In some embodiments, residual blood is removed from the placenta before cell harvest. This may be done by a variety of methods known to those skilled in the art, for example by perfusion. In this context, the term “perfuse” or “perfusion” refers to the act of pouring or passaging a fluid over or through an organ or tissue. In certain embodiments, the placental tissue may be from any mammal, while in other embodiments, the placental tissue is human. A convenient source of placental tissue is a post-partum placenta (e.g., less than 48 hours after birth); however, a variety of sources of placental tissue or cells may be contemplated by the skilled person. In other embodiments, the placenta is used within 36, 30, 28, 24, 20, 16, 14, 12, 10, 8, 6, 5, 4, 3, 2, or 1 hour of birth. In certain embodiments, particularly when the waiting period is over 4 hours, the placenta is kept chilled prior to harvest of the cells, in some embodiments in an antibiotic-containing buffer. In other embodiments, prepartum placental tissue is used. Such tissue may be obtained, for example, from a chorionic villus sampling or by other methods known in the art. Once placental cells are obtained, they are, in certain embodiments, allowed to adhere to the surface of an adherent material to thereby isolate adherent cells. In some embodiments, the donor is 35 years old or younger, while in other embodiments, the donor may be any woman of childbearing age.

As mentioned, in some embodiments the source of the ASC is a non-fetal source, for example maternal cells from the placenta or somatic tissue from a pediatric or adult donor, for example adipose tissue, bone marrow, peripheral blood, umbilical cord blood, synovial fluid, synovial membranes, and ligaments such as the periodontal ligament. Those skilled in the art will appreciate in light of the present disclosure that ASC may be extracted from various body tissues, using standard techniques such as physical and/or enzymatic tissue disruption, in some embodiments followed by marker-based cell sorting, and then may be subjected to the culturing methods described herein.

As mentioned, the ASC are, in some embodiments, derived from adipose tissue. The phrase “adipose tissue” refers to a connective tissue that comprises fat cells (adipocytes). Adipose tissue-derived ASC may be extracted, in various embodiments, by a variety of methods known to those skilled in the art, for example those described in U.S. Pat. Nos. 9,200,255, 9,249,393, and 6,153,432, which are incorporated herein by reference. The adipose tissue may be derived, in other embodiments, from omental/visceral, mammary, gonadal, or other adipose tissue sites. In some embodiments, the adipose can be isolated by liposuction.

In other embodiments, ASC may be derived from adipose tissue by treating the tissue with a digestive enzyme (non-limiting examples of which are collagenase, trypsin, dispase, hyaluronidase or DNAse); and ethylenediaminetetra-acetic acid (EDTA). The cells may be, in some embodiments, subjected to physical disruption, for example using a nylon or cheesecloth mesh filter. In other embodiments, the cells are subjected to differential centrifugation directly in media or over a Ficoll™ or Percoll™ or other particulate gradient (see U.S. Pat. No. 7,078,230, which is incorporated herein by reference).

In certain embodiments, the ASC that are subsequently expanded are mesenchymal stromal cells (MSC). These cells may, in some embodiments, be isolated from many adult tissues, such as placenta, bone marrow and adipose. In further embodiments, the cells are human MSC as defined by The Mesenchymal and Tissue Stem Cell Committee of the International Society for Cellular Therapy (Dominici et al, 2006), based on the following 3 criteria: 1. Plastic-adherence when maintained in standard culture conditions (a minimal essential medium plus 20% fetal bovine serum (FBS)). 2. Expression of the surface molecules CD105, CD73 and CD90, and lack of expression of CD45, CD34, CD14 or CD11 b, CD79a or CD19 and HLA-DR. 3. Differentiation into osteoblasts, adipocytes and chondroblasts in vitro. In some embodiments, the cells are bone marrow (BM)-derived MSC, in more specific embodiments human BM-derived MSC.

Alternatively or in addition, the ASC that are subsequently expanded are mesenchymal-like ASC, which exhibit a marker pattern similar to “classical” MSC, but do not differentiate into osteocytes, under conditions where “classical” MSC would differentiate into osteocytes. In other embodiments, the cells exhibit a marker pattern similar to MSC, but do not differentiate into adipocytes, under conditions where MSC would differentiate into adipocytes. In still other embodiments, the cells exhibit a marker pattern similar to MSC, but do not differentiate into either osteocytes or adipocytes, under conditions where MSC would differentiate into osteocytes or adipocytes, respectively. The MSC used for comparison in these assays are, in one embodiment, MSC that have been harvested from BM and cultured in 2D culture. In other embodiments, the MSC used for comparison have been harvested from BM and cultured in 2D culture, followed by 3D culture.

Alternatively or additionally, the ASC that are subsequently expanded may express a marker or a collection of markers (e.g. surface marker) characteristic of MSC or mesenchymal-like stromal cells. Examples of surface markers include but are not limited to CD105 (UniProtKB Accession No. P17813; SEQ ID No. 59), CD29 (UniProtKB Accession No. P05556; SEQ ID No. 60), CD44 (UniProtKB Accession No. P16070; SEQ ID No. 61), CD73 (UniProtKB Accession No. P21589; SEQ ID No. 62), and CD90 (UniProtKB Accession No. P04216; SEQ ID No. 63). Examples of markers expected to be absent from stromal cells are CD3 (UniProtKB Accession Nos. P09693 [gamma chain] P04234 [delta chain], P07766 [epsilon chain], and P20963 [zeta chain][SEQ ID Nos. 64-67]), CD4 (UniProtKB Accession No. P01730; SEQ ID No. 68), CD34 (UniProtKB Accession No. P28906; SEQ ID No. 69), CD45 (UniProtKB Accession No. P08575; SEQ ID No. 70), CD80 (UniProtKB Accession No. P33681; SEQ ID No. 71), CD19 (UniProtKB Accession No. P15391; SEQ ID No. 72), CD5 (UniProtKB Accession No. P06127; SEQ ID No. 73), CD20 (UniProtKB Accession No. P11836; SEQ ID No. 74), CD11B (UniProtKB Accession No. P11215; SEQ ID No. 75), CD14 (UniProtKB Accession No. P08571; SEQ ID No. 76), CD79-alpha (UniProtKB Accession No. B5QTD1; SEQ ID No. 77), and HLA-DR (UniProtKB Accession Nos. P04233 [gamma chain], P01903 [alpha chain], and P01911 [beta chain][SEQ ID Nos. 78-80]). Those skilled in the art will appreciate that the presence of complex antigens such as CD3 and HLA-DR may be detected by antibodies recognizing any of their component parts, such as, but not limited to, those described herein.

In still other embodiments, the cells that are subsequently expanded are a placental cell population that is a mixture of fetal and maternal cells. In more specific embodiments, the mixture contains 20-80% fetal cells; 30-80% fetal cells; 40-80% fetal cells; 50-80% fetal cells; 60-80% fetal cells; 20-90% fetal cells; 30-90% fetal cells; 40-90% fetal cells; 50-90% fetal cells; 60-90% fetal cells; 20-80% maternal cells; 30-80% maternal cells; 40-80% maternal cells; 50-80% maternal cells; 60-80% maternal cells; 20-90% maternal cells; 30-90% maternal cells; 40-90% maternal cells; 50-90% maternal cells; or 60-90% maternal cells.

As will be appreciated by those skilled in the art, a mixture of fetal and maternal cells can be obtained by mincing and/or enzymatically treating a whole placenta, or various parts thereof. In an exemplary, non-limiting protocol, whole, placenta, excluding the amnion and chorion, is minced. Fragments are washed with isotonic buffer, optionally including antibiotics, then incubated for with a proteolytic enzyme (e.g. collagenase) and optionally DNAse in isotonic buffer. Medium (e.g. DMEM), optionally including Glutamine and antibiotics, is added, and cells are filtered and centrifuged. The cells were suspended in culture medium, seeded in flasks, and incubated under conditions compatible with expansion.

In still other embodiments, the cells that are subsequently expanded may be allogeneic, or in other embodiments, the cells may be autologous with regard to the patient. In other embodiments, the cells may be fresh or, in other embodiments, frozen (e.g., cryopreserved).

Additional Method Steps and Characteristics

In other embodiments, the described incubation/expansion steps utilize microcarriers, which may, in certain embodiments, be inside a bioreactor. Microcarriers are known to those skilled in the art, and are described, for example in U.S. Pat. Nos. 8,828,720, 7,531,334, 5,006,467, which are incorporated herein by reference. Microcarriers are also commercially available, for example as Cytodex™ (available from Pharmacia Fine Chemicals, Inc.,) Superbeads (commercially available from Flow Labs, Inc.,), and as DE-52 and DE-53 (commercially available from Whatman, Inc.). In certain embodiments, the microcarriers are packed inside a bioreactor.

In certain embodiments, further steps of purification or enrichment may be performed. Such methods include, but are not limited to, cell sorting using markers for ASC and/or, in various embodiments, MSC or mesenchymal-like stromal cells.

Cell sorting, in this context, refers to any procedure, whether manual, automated, etc., that selects cells on the basis of their expression of one or more markers, their lack of expression of one or more markers, or a combination thereof. Those skilled in the art will appreciate that data from one or more markers can be used individually or in combination in the sorting process.

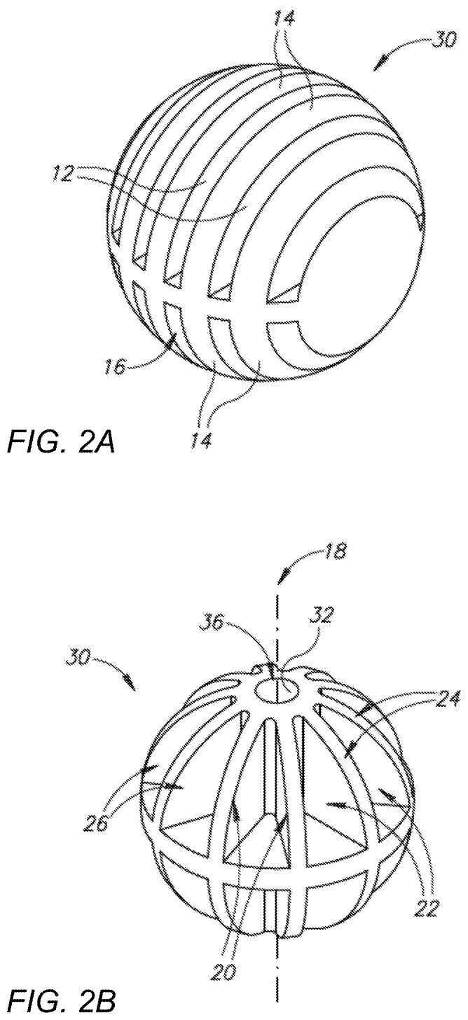

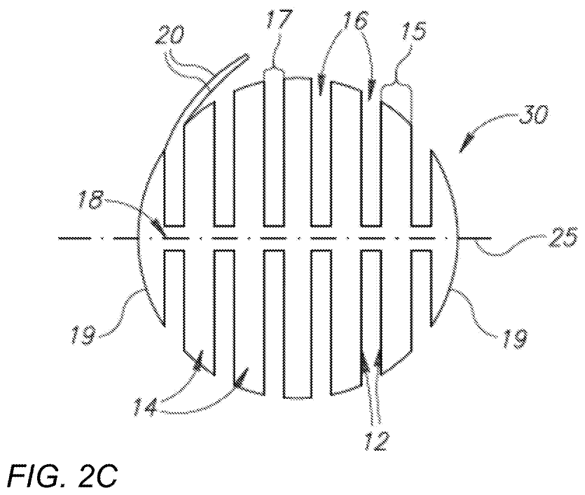

In some embodiments, with reference to FIGS. 2A-B, and as described in WO/2014/037862, published on Mar. 13, 2014, which is incorporated herein by reference in its entirety, grooved carriers 30 are used for proliferation and/or incubation of ASC. In various embodiments, the carriers may be used following a 2D incubation (e.g. on culture plates or dishes), or without a prior 2D incubation. In other embodiments, incubation on the carriers may be followed by incubation on a 3D substrate in a bioreactor, which may be, for example, a packed-bed substrate or microcarriers; or incubation on the carriers may not be followed by incubation on a 3D substrate. Carriers 30 can include multiple two-dimensional (2D) surfaces 12 extending from an exterior of carrier 30 towards an interior of carrier 30. As shown, the surfaces are formed by a group of ribs 14 that are spaced apart to form openings 16, which may be sized to allow flow of cells and culture medium (not shown) during use. With reference to FIG. 2C, carrier 30 can also include multiple 2D surfaces 12 extending from a central carrier axis 18 of carrier 30 and extending generally perpendicular to ribs 14 that are spaced apart to form openings 16, creating multiple 2D surfaces 12. In some embodiments, carriers 30 are “3D bodies” as described in WO/2014/037862; the contents of which relating to 3D bodies are incorporated herein by reference.

In certain embodiments, the described carriers (e.g. grooved carriers) are used in a bioreactor. In some, the carriers are in a packed conformation.

In still other embodiments, the material forming the multiple 2D surfaces comprises at least one polymer. Suitable coatings may, in some embodiments, be selected to control cell attachment or parameters of cell biology.

Harvesting

In certain embodiments, the described method further comprises the subsequent step (following the described incubation(s) in one or more media) of harvesting the expanded cells by removing them from the 3D culture apparatus. In more particular embodiments, cells may be removed from a 3D matrix while the matrix remains within the bioreactor.

In certain embodiments, the harvest from the bioreactor is performed when at least about 10%, 12%, 14%, 16%, 18%, 20%, 22%, 24%, 26%, 28%, or in other embodiments at least 30%, of the cells are in the S and G2/M phases (collectively), as can be assayed by various methods known in the art, for example FACS detection. Typically, in the case of FACS, the percentage of cells in S and G2/M phase is expressed as the percentage of the live cells, after gating for live cells, for example using a forward scatter/side scatter gate. Those skilled in the art will appreciate that the percentage of cells in these phases correlates with the percentage of proliferating cells.

In still other embodiments, the expanded cells are harvested from the bioreactor by a process comprising vibration or agitation, for example as described in PCT International Application Publ. No. WO 2012/140519, which is incorporated herein by reference. In certain embodiments, during harvesting, the cells are agitated at 0.7-6 Hertz, or in other embodiments 1-3 Hertz, during, or in other embodiments during and after, treatment with a protease, optionally also comprising a calcium chelator. In certain embodiments, the carriers containing the cells are agitated at 0.7-6 Hertz, or in other embodiments 1-3 Hertz, while submerged in a solution or medium comprising a protease, optionally also comprising a calcium chelator. Non-limiting examples of a protease plus a calcium chelator are trypsin, or another enzyme with similar activity, optionally in combination with another enzyme, non-limiting examples of which are Collagenase Types I, II, III, and IV, with EDTA. Enzymes with similar activity to trypsin are well known in the art; non-limiting examples are TrypLE™, a fungal trypsin-like protease, and Collagenase, Types I, II, III, and IV, which are available commercially from Life Technologies. Enzymes with similar activity to collagenase are well known in the art; non-limiting examples are Dispase I and Dispase II, which are available commercially from Sigma-Aldrich. In still other embodiments, the cells are harvested by a process comprising an optional wash step, followed by incubation with collagenase, followed by incubation with trypsin. In various embodiments, at least one, at least two, or all three of the aforementioned steps comprise agitation. In more specific embodiments, the total duration of agitation during and/or after treatment with protease plus a calcium chelator is between 2-10 minutes, in other embodiments between 3-9 minutes, in other embodiments between 3-8 minutes, and in still other embodiments between 3-7 minutes. In still other embodiments, the cells are subjected to agitation at 0.7-6 Hertz, or in other embodiments 1-3 Hertz, during the wash step before the protease and calcium chelator are added.

Those skilled in the art will appreciate that a variety of isotonic buffers may be used for washing cells and similar uses. Hank's Balanced Salt Solution (HBSS; Life Technologies) is only one of many buffers that may be used.

It will be appreciated that additional components may be added to the culture medium. Such components may be antibiotics, antimycotics, albumin, amino acids, and other components known to the art for the culture of cells.

The various media described herein, i.e. the serum-free medium and the serum-containing medium, may be independently selected from each of the described embodiments relating to medium composition. In certain embodiments, the only difference between the 2 media is the presence of the added serum. In other embodiments, the 2 growth media differ in other respects.

Characteristics of Expanded Cells

Following expansion by the described methods, in certain embodiments the majority, in other embodiments over 60%, over 70%, over 80%, or over 90% of the expanded cells are positive for one or more of CD29, CD73, CD90, and CD105; in other embodiments, 2 or more of these markers; in other embodiments, all 4 of these markers. In other embodiments, over 80% of the expanded cells are positive for CD29, CD73, CD90, and CD105; and less than 50% of the cells are positive for CD49. In yet other embodiments, less than 20%, 15%, or 10% of the described cells are positive for CD34, CD39, CD56, and CD106. In various embodiments, the cell population may be less than 50%, less than 40%, less than 30%, less than 20%, less than 10%, more than 50%, more than 40%, more than 30%, more than 20%, or more than 10% positive for CD200. Alternatively or in addition over 90% of said expanded ASC population do not express at least one of CD3, CD4, CD34, CD39, and CD106; in other embodiments, 2 or more of these markers; in other embodiments, all 4 of these markers. In certain embodiments, the cells are placenta-derived, while in other embodiments, the cells are derived from adipose tissue; from BM; or from other sources.

“Positive” expression of a marker indicates a value higher than the range of the main peak of a fluorescence-activated cell sorting (FACS) isotype control histogram; this term is synonymous herein with characterizing a cell as “express”/“expressing” a marker. Positive expression of a combination of markers by a percentage of a population denotes that indicated percentage of cells in the population are individual cells expressing the markers in combination. “Negative” expression of a marker indicates a value falling within the range of the main peak of an isotype control histogram; this term is synonymous herein with characterizing a cell as “not express”/“not expressing” a marker. Negative expression of a combination of markers by a percentage of a population denotes that indicated percentage of cells in the population are individual cells expressing none of the mentioned markers.

In other embodiments, each of CD29, CD73, CD90, and CD105 is expressed by more than 80% of the cells that have been expanded; and over 90% (or in other embodiments, over 95%, or in other embodiments, over 98%) of the cells do not differentiate into osteocytes, after incubation for 17 days with a solution containing 0.1 mcM dexamethasone, 0.2 mM ascorbic acid, and 10 mM glycerol-2-phosphate, in plates coated with vitronectin and collagen (“standard osteogenesis induction conditions”). In yet other embodiments, each of CD34, CD39, CD56, and CD106 is expressed by less than 10% of the cells; and the cells do not differentiate into osteocytes, after incubation under the aforementioned conditions. In other embodiments, each of CD29, CD73, CD90, and CD105 is expressed by more than 90% of the cells, each of CD34, CD39, CD56, and CD106 is expressed by less than 5% of the cells; and the cells do not differentiate into osteocytes, after incubation under the aforementioned conditions. In still other embodiments, the conditions are incubation for 26 days with a solution containing 10 mcM dexamethasone, 0.2 mM ascorbic acid, 10 mM glycerol-2-phosphate, and 10 nM Vitamin D, in plates coated with vitronectin and collagen (“modified osteogenesis induction conditions”). The aforementioned solutions will typically contain cell culture medium such as DMEM+10% serum or the like, as will be appreciated by those skilled in the art. In certain embodiments, the cells are placenta-derived, while in other embodiments, the cells are derived from adipose tissue; from BM; or from other sources.

In other embodiments, each of CD29, CD73, CD90, and CD105 is expressed by more than 80% of the cells that have been expanded; and over 90% (or in other embodiments, over 95%, or in other embodiments, over 98%) of the cells do not differentiate into adipocytes, after incubation in adipogenesis induction medium, namely a solution containing 1 mcM dexamethasone, 0.5 mM 3-Isobutyl-1-methylxanthine (IBMX), 10 mcg/mL Insulin, and 100 mcM indomethacin, on days 1, 3, 5, 9, 11, 13, 17, 19, and 21; and replacement of the medium with adipogenesis maintenance medium, namely a solution containing 10 mcg/mL Insulin, on days 7 and 15, for a total of 25 days (“standard adipogenesis induction conditions”). In yet other embodiments, each of CD34, CD39, CD56, and CD106 is expressed by less than 10% of the cells; and the cells do not differentiate into adipocytes, after incubation under the aforementioned conditions. In other embodiments, each of CD29, CD73, CD90, and CD105 is expressed by more than 90% of the cells, each of CD34, CD39, CD56, and CD106 is expressed by less than 5% of the cells; and the cells do not differentiate into adipocytes, after incubation under the aforementioned conditions. In still other embodiments, a modified adipogenesis induction medium, containing 1 mcM dexamethasone, 0.5 mM IBMX, 10 mcg/mL Insulin, and 200 mcM indomethacin is used, and the incubation is for a total of 26 days (“modified adipogenesis induction conditions”). In still other embodiments, over 90% of the cells in each population do not differentiate into either adipocytes or osteocytes under the aforementioned standard conditions. In yet other embodiments, over 90% of the cells in each population do not differentiate into either adipocytes or osteocytes under the aforementioned modified conditions. The aforementioned solutions will typically contain cell culture medium such as DMEM+10% serum or the like, as will be appreciated by those skilled in the art. In yet other embodiments, the majority, in other embodiments over 70%, or in other embodiments over 80%, over 90%, or over 95% of the ASC do not differentiate into adipogenic cells under any of the aforementioned conditions. In certain embodiments, the cells are placenta-derived, while in other embodiments, the cells are derived from adipose tissue; from BM; or from other sources.

Additionally or alternatively, the cells that have been expanded secrete or express IL-6, IL-8 (UniProt identifier P10145; SEQ ID No. 81), eukaryotic translation elongation factor 2 (EEEF2), reticulocalbin 3, EF-hand calcium binding domain (RCN₂), and/or calponin 1 basic smooth muscle (CNN1). In more specific embodiments, greater than 50%, in other embodiments greater than 55%, in other embodiments greater than 60%, in other embodiments greater than 65%, in other embodiments greater than 70%, in other embodiments greater than 75%, in other embodiments greater than 80%, in other embodiments greater than 85%, in other embodiments greater than 90%, in other embodiments greater than 95%, in other embodiments greater than 96%, in other embodiments greater than 97%, in other embodiments greater than 98%, in other embodiments greater than 99%, of the cells express or secrete at least one, in other embodiments at least 2, in other embodiments at least 3, in other embodiments at least 4, in other embodiments all five of the aforementioned proteins. In certain embodiments, the cells are placenta-derived, while in other embodiments, the cells are derived from adipose tissue; from BM; or from other sources.

A cell is said to express a protein or factor if the presence of protein or factor is detectable by standard methods, an example of which is a detectable signal using fluorescence-activated cell sorting (FACS), relative to an isotype control. Reference herein to “secrete”/“secreting”/“secretion” relates to a detectable secretion of the indicated factor, above background levels in standard assays. For example, 0.5×10⁶ ASC can be suspended in 4 mL medium (DMEM+10% fetal bovine serum (FBS)+2 mM L-Glutamine), added to each well of a 6 well-plate, and cultured for 24 hrs in a humidified incubator (5% CO₂, at 37° C.). After 24 h, DMEM is removed, and cells are cultured for an additional 24 hrs in 1 mL RPMI 1640 medium+2 mM L-Glutamine+0.5% HSA. The CM is collected from the plate, and cell debris is removed by centrifugation.

In other embodiments, the described expanded cells exhibit a spindle shape when cultured in 2D culture.

In certain embodiments, the expanded cells have been transfected with one or more therapeutic factors, which may be, in certain embodiments, pro-angiogenic or anti-inflammatory factors. In other embodiments, the cells have not been transfected with any exogenous genetic material.

In still other embodiments, the population of expanded cells is a fetal cell population. In other embodiments, the expanded population is a mixture of fetal and maternal cells, which is predominant fetal cells. In more specific embodiments, the mixture contains 70-100% fetal cells; 80-100% fetal cells; 85-100% fetal cells; 90-100% fetal cells; 92-100% fetal cells; 95-100% fetal cells; 96-100% fetal cells; 97-100% fetal cells; 98-100% fetal cells; 99-100% fetal cells or 99.5-100% fetal cells. In other embodiments, the cells are substantially entirely fetal cells. “Substantially entirely”, in this context, refers to a lack of detectable presence of maternal cell by standard fluorescence-activated cell sorting assays.

In still other embodiments, the population of expanded cells is a maternal cell population. In other embodiments, the expanded population is a mixture of fetal and maternal cells, which is predominant maternal cells. In more specific embodiments, the mixture contains 70-100%, 80-100%, 85-100%, 90-100%, 92-100%, 95-100%, 96-100%, 97-100%, 98-100%, 99-100%, or 99.5-100% maternal cells. In other embodiments, the cells are substantially entirely maternal cells. “Substantially entirely”, in this context, refers to a lack of detectable presence of fetal cells by standard fluorescence-activated cell sorting assays.

In other embodiments, the described cells produce or secrete elevated amounts of therapeutic factors, relative to untreated cells. In certain embodiments, the therapeutic factors are one or more anti-inflammatory factors, or, in other embodiments, pro-inflammatory factors, or, in other embodiments, angiogenic factors.

Cells, CM, Compositions, and Methods of Utilizing Same

In other embodiments, there is provided a population of cells expanded by the described methods. In other embodiments, there is provided a composition, comprising the cells. In certain embodiments, the composition is a pharmaceutical composition and/or further comprises a pharmacologically acceptable excipient. In further embodiments, the excipient is an osmoprotectant or cryoprotectant, an agent that protects cells from the damaging effect of freezing and ice formation, which may in some embodiments be a permeating compound, non-limiting examples of which are dimethyl sulfoxide (DMSO), glycerol, ethylene glycol, formamide, propanediol, poly-ethylene glycol, acetamide, propylene glycol, and adonitol; or may in other embodiments be a non-permeating compound, non-limiting examples of which are lactose, raffinose, sucrose, trehalose, and d-mannitol. In other embodiments, both a permeating cryoprotectant and a non-permeating cryoprotectant are present. In other embodiments, the excipient is a carrier protein, a non-limiting example of which is albumin. In still other embodiments, both an osmoprotectant and a carrier protein are present; in certain embodiments, the osmoprotectant and carrier protein may be the same compound. Alternatively or in addition, the composition is frozen. The cells may be any embodiment of expanded cells mentioned herein, each of which is considered a separate embodiment.

In other embodiments, there is provided conditioned medium (CM) derived from the described methods, for example post-incubation medium from the described first or subsequent incubation step. In still other embodiments, there is provided CM derived from incubating cells following expansion by the described methods. In yet other embodiments, there is provided a pharmaceutical composition comprising the CM. Those skilled in the art will appreciate that, in certain embodiments, various bioreactors may be used to prepare CM, including but not limited to plug-flow bioreactors, and stationary-bed bioreactors (Kompier R et al. Use of a stationary bed reactor and serum-free medium for the production of recombinant proteins in insect cells. Enzyme Microb Technol. 1991. 13(10):822-7.) For example, CM is produced as a by-product of the described methods for cell expansion. The CM in the bioreactor can be removed from the bioreactor or otherwise isolated. In other embodiments, the described expanded cells are removed from the bioreactor and incubated in another apparatus (a non-limiting example of which is a tissue culture apparatus), and CM from the cells is collected.

In still other embodiments, there is provided a culture, comprising the described cells, or in other embodiments a bioreactor, comprising the described culture. Except where indicated otherwise, the term “bioreactor” refers to an apparatus comprising a cell culture chamber and external medium reservoir (a non-limiting example of which is a feed bag) that is operably connected with the cell culture chamber so as to enable medium exchange between the two compartments (perfusion). The term excludes decellularized organs and tissues derived from a living being. In some embodiments, the bioreactor further comprises a synthetic material that is a 3D substrate. The cells may be any embodiment of expanded cells mentioned herein, each of which is considered a separate embodiment.

In still other embodiments, there is provided a suspension comprising any of the described cell populations. In certain embodiments, the suspension comprises a pharmaceutically acceptable excipient. In other embodiments, the suspension is a pharmaceutical composition. In still other embodiments, the suspension is frozen and further comprises, in some embodiments, a cryoprotectant. In other embodiments is provided a composition, comprising the suspension. In certain embodiments, the composition further comprises a pharmacologically acceptable excipient. In further embodiments, the excipient is a cryopreservant (cryoprotectant), or is a carrier protein. Alternatively or in addition, the composition is frozen. Each of the aforementioned cell populations represents a separate embodiment in this regard.

In certain embodiments, the described pharmaceutical composition is indicated for enhancing repopulation of HSC.

In various embodiments, the described cells are able to exert the described therapeutic effects, each of which is considered a separate embodiment, with or without the cells themselves engrafting in the host. For example, the cells may, in various embodiments, be able to exert a therapeutic effect, without themselves surviving for more than 3 days, more than 4 days, more than 5 days, more than 6 days, more than 7 days, more than 8 days, more than 9 days, more than 10 days, or more than 14 days.

Also provided herein are extracellular vesicles, e.g. exosomes, secreted by the described expanded cells. Methods of isolating extracellular vesicles are known in the art, and include, for example, immuno-magnetic isolation, for example as described in Clayton A et al, 2001; Mathias R A et al, 2009; and Crescitelli R et al, 2013.

In some embodiments, the vesicles are harvested from a bioreactor in which the cells have been incubated. Alternatively or in addition, the cells are cryopreserved, and then are thawed, after which the vesicles are isolated. In some embodiments, after thawing, the cells are cultured in 2D culture, from which the vesicles are harvested.

The described cells, CM, or exosomes can be, in some embodiments, administered as a part of a pharmaceutical composition that further comprises one or more pharmaceutically acceptable carriers. Hereinafter, the term “pharmaceutically acceptable carrier” refers to a carrier or a diluent. In some embodiments, the carrier or diluent does not cause significant irritation to a subject and does not abrogate the biological activity and properties of the administered cells. Examples, without limitations, of carriers are propylene glycol, saline, emulsions and mixtures of organic solvents with water. In some embodiments, the pharmaceutical carrier is an aqueous solution of saline. In other embodiments, the composition further comprises a pharmacologically acceptable excipient. In further embodiments, the excipient is a cryoprotectant, or is a carrier protein. Alternatively or in addition, the composition is frozen.

In other embodiments, compositions are provided herein that comprises cells or CM in combination with an excipient, e.g., a pharmacologically acceptable excipient. In further embodiments, the excipient is an osmoprotectant or cryoprotectant, an agent that protects cells from the damaging effect of freezing and ice formation, which may in some embodiments be a permeating compound, non-limiting examples of which are dimethyl sulfoxide (DMSO), glycerol, ethylene glycol, formamide, propanediol, poly-ethylene glycol, acetamide, propylene glycol, and adonitol; or may in other embodiments be a non-permeating compound, non-limiting examples of which are lactose, raffinose, sucrose, trehalose, and d-mannitol. In other embodiments, both a permeating cryoprotectant and a non-permeating cryoprotectant are present. In other embodiments, the excipient is a carrier protein, a non-limiting example of which is albumin. In still other embodiments, both an osmoprotectant and a carrier protein are present; in certain embodiments, the osmoprotectant and carrier protein may be the same compound. Alternatively or in addition, the composition is frozen. The cells may be any embodiment of expanded cells mentioned herein, each of which is considered a separate embodiment.

Since non-autologous cells may in some cases induce an immune reaction when administered to a subject, several approaches may be utilized according to the methods provided herein to reduce the likelihood of rejection of non-autologous cells. In some embodiments, these approaches include either suppressing the recipient immune system or encapsulating the non-autologous cells in immune-isolating, semipermeable membranes before transplantation. In some embodiments, this may be done whether or not the cells themselves engraft in the host. For example, the majority of the cells may, in various embodiments, not survive after engraftment for more than 3 days, more than 4 days, more than 5 days, more than 6 days, more than 7 days, more than 8 days, more than 9 days, more than 10 days, or more than 14 days.

In other embodiments, an immunosuppressive agent is present in the pharmaceutical composition. Examples of immunosuppressive agents that may be used in the method and compositions provided herein include, but are not limited to, methotrexate, cyclophosphamide, cyclosporine, cyclosporine A, chloroquine, hydroxychloroquine, sulfasalazine (sulphasalazopyrine), gold salts, D-penicillamine, leflunomide, azathioprine, anakinra, infliximab (REMICADE), etanercept, TNF-alpha blockers, biological agents that antagonize one or more inflammatory cytokines, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, and tramadol.

One may, in various embodiments, administer the pharmaceutical composition in a systemic manner (as detailed hereinabove). Alternatively, one may administer the pharmaceutical composition locally, for example, via injection of the pharmaceutical composition directly into an affected tissue region of a patient. In other embodiments, the cells are administered intravenously (IV), intravascularly, subcutaneously (SC), or intraperitoneally (IP), each of which is considered a separate embodiment. In still other embodiments, the pharmaceutical composition is administered intralymphatically, for example as described in U.S. Pat. No. 8,679,834 in the name of Eleuterio Lombardo and Dirk Buscher, which is hereby incorporated by reference.

In other embodiments, for injection, the described cells may be formulated in aqueous solutions, e.g. in physiologically compatible buffers such as Hank's solution, Ringer's solution, or physiological salt buffer, optionally in combination with medium containing cryopreservation agents.

For any preparation used in the described methods, the therapeutically effective amount or dose can be estimated initially from in vitro and cell culture assays. Often, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals.

The data obtained from these in vitro and cell culture assays and animal studies can be used in formulating a range of dosage for use in human. The dosage may vary depending upon the dosage form employed and the route of administration utilized. The exact formulation, route of administration and dosage can be, in some embodiments, chosen by the individual physician in view of the patient's condition.

Depending on the severity and responsiveness of the condition to be treated, dosing can be of a single or, in other embodiments, a plurality of administrations, with a course of treatment lasting from several days to several weeks or, in other embodiments, until alleviation of the disease state is achieved.

In certain embodiments, following administration, the majority of the cells, in other embodiments more than a percentage selected from 60%, 70%, 80%, 90%, 95%, 96%, 97%, 98%, and 99% of the cells are no longer detectable within the subject 1 month after administration.

Compositions including the described preparations formulated in a compatible pharmaceutical carrier may also be prepared, placed in an appropriate container, and labeled for treatment of an indicated condition.

The described compositions may, if desired, be packaged in a container that is accompanied by instructions for administration. The container may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

The described cells are, in some embodiments, suitably formulated as pharmaceutical compositions which can be suitably packaged as an article of manufacture. Such an article of manufacture comprises a packaging material which comprises a label describing a use in treating an immune-mediated or circulatory disorder, as described herein. In other embodiments, a pharmaceutical agent is contained within the packaging material, wherein the pharmaceutical agent is effective for the treatment of a hematologic disorder. In some embodiments, the pharmaceutical composition is frozen.

A typical dosage of the described cells used alone might range, in some embodiments, from about 10 million-500 million cells per administration. For example, the dosage can be, in some embodiments, 10, 20, 30, 40, 50, 60, 70, 80, 90, 100, 125, 150, 175, 200, 225, 250, 275, 300, 325, 350, 375, 400, 425, 450, 475, or 500 million cells or any amount in between these numbers. It is further understood that a range of ASC can be used including from about 10 to about 500 million cells, from about 100 to about 400 million cells, from about 150 to about 300 million cells. Accordingly, disclosed herein are therapeutic methods, the method comprising administering to a subject a therapeutically or prophylactically effective amount of cells, wherein the dosage administered to the subject is 10, 20, 30, 40, 50, 60, 70, 80, 90, 100, 125, 150, 175, 200, 225, 250, 275, 300, 325, 350, 375, 400, 425, 450, 475, or 500 million cells or, in other embodiments, between 150 million to 300 million cells. Cells, compositions comprising cells, and/or medicaments manufactured using cells can be administered, in various embodiments, in a series of 1, 2, 3, 4, 5, 6, 7, 8, 9, 10, 11, 12, 13, 14, 15, 1-10, 1-15, 1-20, 2-10, 2-15, 2-20, 3-20, 4-20, 5-20, 5-25, 5-30, 5-40, or 5-50 injections, or more.

In other embodiments is provided a method of enhancing repopulation of HSC in a subject in need thereof, comprising administering to the subject a pharmaceutical composition comprising the described exosomes. Also provided is a composition for treating or inhibiting transplant rejection, comprising the described exosomes. Provided in addition is use of the described exosomes in the preparation of a medicament for treating or inhibiting transplant rejection.

Also provided herein is a method of enhancing repopulation of hematopoietic stem cells (HSC) in a subject in need thereof, comprising administering to the subject a pharmaceutical composition comprising the described expanded cells. Also provided is a composition for enhancing HSC engraftment in a subject in need thereof, comprising the described cells.

Provided in addition is use of the described cells in the preparation of a medicament for enhancing HSC engraftment. Those skilled in the art will appreciate in light of the present disclosure that the described cells are useful for enhancing HSC engraftment, which may, in more specific embodiments, be following radiation-induced bone marrow ablation, chemotherapy, or accidental radiation exposure.

Also provided herein is a method of enhancing engraftment of exogenous HSC in a subject in need thereof, comprising administering to the subject a pharmaceutical composition comprising the described expanded cells. Also provided is a composition for enhancing exogenous HSC engraftment in a subject in need thereof, comprising the described expanded cells. Provided in addition is use of the described expanded cells in the preparation of a medicament for enhancing exogenous HSC engraftment. Those skilled in the art will appreciate in light of the present disclosure that the described cells are useful for enhancing exogenous HSC engraftment, for example decreasing the time of engraftment and/or the number of cells needed for successful engraftment. Those skilled in the art will appreciate that relatively small numbers of cells obtained, for example with umbilical cord blood transplantation, may limit the effectiveness of the procedure. The treatment may, in more specific embodiments, follow radiation-induced bone marrow ablation, chemotherapy, or accidental radiation exposure.

In certain embodiments, the cells are able to support repopulation of the recipient's endogenous hematopoietic system without further co-transplantation of HSC, or in other embodiments are able to enhance exogenous HSC engraftment. This can be achieved, in various embodiments, with or without the cells themselves engrafting in the host. For example, the cells may, in various embodiments, be able to support repopulation of red blood cells, white blood cells, and/or platelets, without themselves surviving for more than 3 days, more than 4 days, more than 5 days, more than 6 days, more than 7 days, more than 8 days, more than 9 days, more than 10 days, or more than 14 days.

Also provided herein is a method of treating incomplete engraftment of a hematopoietic stem cell (HSC) transplant in a subject in need thereof, comprising administering to the subject a pharmaceutical composition comprising the described expanded cells.

Also provided herein is a method of enhancing hematopoiesis in a subject having received an RIC HSC transplant in a subject in need thereof, comprising administering to the subject a pharmaceutical composition comprising the described expanded cells.

Also provided herein is a method of treating myelodysplastic syndrome (MDS) in a subject in need thereof, comprising administering to the subject a pharmaceutical composition comprising the described expanded cells.

Also provided herein is a method of treating failed engraftment of an HSC transplant in a subject in need thereof, comprising administering to the subject a pharmaceutical composition comprising the described expanded cells.

Also provided herein is a method of enhancing hematopoiesis in a subject having received an HSC transplant after non-myeloablative conditioning in a subject in need thereof, comprising administering to the subject a pharmaceutical composition comprising the described expanded cells.

Also provided herein is a method of treating bone marrow deficiency in a subject in need thereof, comprising administering to the subject a pharmaceutical composition comprising the described expanded cells.

Also provided herein is a method of treating a subject following exposure to radiation, comprising administering to the subject a pharmaceutical composition comprising the described expanded cells, thereby treating a subject following exposure to radiation.

Also provided herein is a method of treating a subject following chemotherapy, comprising administering to the subject a pharmaceutical composition comprising the described expanded cells, thereby treating a subject following chemotherapy.

Also provided herein is a method of treating a subject with a compromised endogenous hematopoietic system, comprising administering to the subject a pharmaceutical composition comprising the described expanded cells, thereby treating a subject with a compromised endogenous hematopoietic system.

According to yet another aspect, there is also provided a method of reducing symptoms associated with radiation sickness or exposure to toxic chemicals, comprising administering to an exposed subject a therapeutically effective amount of the described expanded cells. In some embodiments, the radiation sickness is acute. In some embodiments, the toxic chemicals are administered as part of a chemotherapy. In either of these embodiments, symptoms include, but are not limited to, nausea and vomiting, diarrhea, headache, fever, weight loss, neurological symptoms (e.g., cognitive impairment, seizures, tremor, ataxia, lethargy), leukopenia, anemia, thrombocytopenia, fatigue, weakness, purpura, hemorrhage, epilation, and shock. In some embodiments, the radiation or chemotherapy results in damage to the respiratory system, damage to the nervous system, damage to the gastrointestinal system, damage to the cardiovascular system, damage to the skin, or damage to the renal system.

According to yet another aspect, there is also provided a method of treating acute radiation syndrome (ARS), in a subject in need thereof, comprising administering to an exposed subject a therapeutically effective amount of the described expanded cells.

In other embodiments is provided a method of enhancing engraftment of exogenous HSC, or in other embodiments supporting repopulation of an endogenous hematopoietic system, in a subject in need thereof, comprising administering to the subject a pharmaceutical composition comprising the described exosomes. Also provided is a composition for enhancing engraftment of exogenous HSC, or in other embodiments supporting repopulation of an endogenous hematopoietic system, comprising the described exosomes. Provided in addition is use of the described exosomes in the preparation of a medicament for enhancing engraftment of exogenous HSC, or in other embodiments supporting repopulation of an endogenous hematopoietic system.

In still other embodiments, there is a provided a method of treating an ischemic disorder, comprising administering to the subject a pharmaceutical composition comprising the described expanded cells. In certain embodiments, the ischemic disorder is a peripheral artery disease (PAD). Alternatively or in addition, the ASC secrete a factor(s) that stimulates angiogenesis. In certain embodiments, the ischemic disorder is critical limb ischemia (CLI). In other embodiments, the ischemic disorder is intermittent claudication (IC). In still other embodiments, the ischemic disorder is selected from ischemia of the central nervous system (CNS) (e.g. ischemic stroke), ischemic heart disease and ischemic renal disease. Other relevant embodiments are described in WO 2009/037690, which is incorporated herein by reference.

In still other embodiments, there is a provided a method of treating an inflammatory disorder, comprising administering to the subject a pharmaceutical composition comprising the described expanded cells. In certain embodiments, the inflammatory disorder is selected from systemic lupus erythematosus (SLE), rheumatoid arthritis, systemic sclerosis, Sjorgen's syndrome, multiple sclerosis (MS), Myasthenia Gravis (MG), Guillain-Barre Syndrome, Hashimoto's Thyroiditis (HT), Graves's Disease, Insulin dependent Diabetes Mellitus (IDDM), and Inflammatory Bowel Disease. Alternatively or in addition, the ASC secrete immunoregulatory and/or anti-inflammatory factor(s). Other relevant embodiments are described in WO/2007/108003, which is incorporated herein by reference.

In still other embodiments, there is a provided a method of treating a neurodegenerative disorder, comprising administering to the subject a pharmaceutical composition comprising the described expanded cells. In some embodiments, the neurodegenerative disorder is selected from Alzheimer's disease, Parkinson's disease, ALS, and Huntington's disease.

In still other embodiments, there is a provided a method of treating a neoplasm or neoplastic disorder in a subject in need thereof, comprising administering to the subject a pharmaceutical composition comprising the described expanded cells. In some embodiments, the neoplastic disorder is selected from the tumors and neoplasms described in WO 2017/141181, which is incorporated herein by reference. In other embodiments, the neoplastic disorder is triple-negative breast cancer (TNBC).

It is clarified that each embodiment of the described expanded cells, and methods of expanding the cells, may be freely combined with each embodiment relating to a therapeutic method or pharmaceutical composition. Furthermore, the cells utilized in the method or contained in the composition can be, in various embodiments, autologous, allogeneic, or xenogenic to the treated subject. Each type of cell may be freely combined with the therapeutic embodiments mentioned herein.

Furthermore, each embodiment of the described exosomes may be freely combined with each embodiment relating to a therapeutic method or pharmaceutical composition.

In still other embodiments, the herein-described CM is used in one or more of the herein-described therapeutic methods. Each embodiment of CM may be freely combined with each embodiment relating to a therapeutic method or pharmaceutical composition.

Subjects and Routes of Administration

In certain embodiments, the subject treated by the described methods and compositions is a human. In other embodiments, the subject may be an animal. In certain embodiments, the subject may be administered with additional therapeutic agents or cells.

In certain embodiments, the described cells or composition comprising same is administered intramuscularly, subcutaneously, or systemically. In this regard, “intramuscular” administration refers to administration into the muscle tissue of a subject; “subcutaneous” administration refers to administration just below the skin; and “intravenous” administration refers to administration into a vein of a subject.

Also disclosed herein are kits and articles of manufacture that are drawn to reagents that can be used in practicing the methods disclosed herein. The kits and articles of manufacture can include any reagent or combination of reagent discussed herein or that would be understood to be required or beneficial in the practice of the disclosed methods, including ASC. In another aspect, the kits and articles of manufacture may comprise a label, instructions, and packaging material, for example for treating a disorder, e.g. an ischemic disorder, a hematopoietic disorder, a neurodegenerative disorder, an inflammatory disorder, or a neoplasm, or for another therapeutic indication mentioned herein.

Additional objects, advantages, and novel features of the invention will become apparent to one ordinarily skilled in the art upon examination of the following examples, which are not intended to be limiting. Additionally, each of the various embodiments and aspects of the invention as delineated hereinabove and as claimed in the claims section below finds experimental support in the following examples.

### EXAMPLES

Reference is now made to the following Examples, which together with the above descriptions illustrate certain embodiments in a non-limiting fashion.

### Example 1: Serum-Free Activation of Expanded Placental Cells Methods

### CM Collection

8×10⁵ ASC were seeded in 4 mL DMEM+20% FBS, in a 6-well plate wells coated with Fibronectin. After 24 hours, the medium was replaced with 1 mL of RPMI 1640 supplemented with 0.5% human albumin, and cells were incubated for an additional 24 hours, after which the conditioned medium was collected

### Bone Marrow Migration (BMM)

1×10⁶ mouse bone marrow (BM) cells per well were seeded into the upper section of a 24 Transwell® plate, and CM was added to the lower section. The apparatus was incubated for 24 hours at 37° C. Migration of the BM cells was assessed by counting the collected BM cells from the lower section with a Vi-Cell® XR counting device. RPMI medium+0.5% human albumin and a reference batch served as negative and positive controls, respectively. Results are presented as Fold to NC (negative control) and calculated by dividing the test result of the tested sample by the negative control result.

### Cytokine Assays

Cytokine concentrations were quantified using ready-to-use kits from R&D Systems, IL-6 (catalog # D6050), IL-8 ((Catalog It D8000C) and MCP-1 (Catalog # DCP00).

### Results

Placental ASC were expanded and activated in culture dishes. Conditions were either (a) Nutristem® basal medium (NBM)+supplement, then activated with 20% FBS; or (b) NBM+ defined factors, followed by activation with 25 ng/mL TNF-alpha and 10 ng/mL IL-1-beta. Cells were compared to a reference batch (positive control), which was expanded in NBM+ supp, in flasks, followed by a bioreactor, followed by activation with FBS in a bioreactor. Population doubling times were comparable between groups (a) and (b). Conditioned media (CM) from the activated cells were tested for cytokine concentrations and ability to induce bone-marrow migration (BMM). This experiment was performed on 2 different batches of placental cells (which were compared to the same reference standard). In both cases, condition (b) produced strong BMM activity and robust cytokine secretion (Tables 1-2).

TABLE 1
Characteristics of cells activated in culture dishes—population 1.

| EM | Activ. | % BMM | % rel. pot. | % to NC | IL-8 | IL-6 | MCP |
|---|---|---|---|---|---|---|---|
| NBM + sup* | FBS | 44 | 100 | 10.4 | 190,000 | 294,000 | 55,000 |
| NBM + sup | FBS | 20 | 22 | 2.3 | 6210 | 370 | 1060 |
| NBM + DF | TN/IL | 26 | 59 | 6.1 | 13,100 | 15,400 | 1200 |

In Tables 1-7, relative potency is measured relative to reference batch, and “% to NC” (where present) “Activ”, indicates percent fold increase over negative control (unstimulated cells). “EM” and for where present, indicate expansion medium and activation factors, respectively; DF stands defined factors; and TN/IL stands for TNF-alpha + IL-1-beta. All cytokine concentrations are expressed in pg/mL. The reference standard, where present, is indicated by an asterisk.

TABLE 2
Characteristics of cells activated in culture dishes—population 2.

| | | % | % | % to | | | |
|---|---|---|---|---|---|---|---|
| EM | Activ. | BMM | rel. pot. | NC | IL-8 | IL-6 | MCP |
| NBM + | FBS | 43 | 100 | 10.6 | 271352 | 68256 | 211129 |
| sup* | | | | | | | |
| NBM + sup | FBS | 30 | 70 | 7.3 | 40,800 | 833 | 50,000 |
| NBM + DF | TN/IL | 28 | 65 | 6.9 | 20844 | 7958 | 8271 |

Similar results were obtained when expanding the cells on tissue culture dishes, followed by further expansion and activation on 3D carriers in a bioreactor. In this case, the experimental groups were either expanded in NBM+(a) supplement or (b) defined factors; and both groups were activated with TNF-alpha and IL-1-beta, as described above. The cells were combined to a reference standard, which was treated with NBM+supp, followed by FBS activation in a bioreactor (Table 3). The bioreactor yields from cells activated with cytokines were superior to the reference standard (Table 4). Similar cytokine secretion results were obtained with EPO and SDF-1.

TABLE 3
Characteristics of cells activated in a bioreactor.

| EM | Activ. | % BMM | % rel. potency | IL-8 | IL-6 |
|---|---|---|---|---|---|
| NBM + sup* | FBS | 36 | 100 | 380,000 | 330,000 |
| NBM + sup | TN/IL | 48 | 107 | 53,700 | 31,500 |
| NBM + DF | TN/IL | 33 | 97 | 326,000 | 284,000 |

TABLE 4
Yield of cells expanded activated in a bioreactor.

| Batch No. | EM | Activ. | Yield (cells/10⁶/gram of carriers) |
|---|---|---|---|
| 1 | NBM + sup | TN/IL | 77.8 |
| 2 | NBM + sup | TN/IL | 112 |
| 3 | NBM + sup | TN/IL | 74.5 |
| 4 | NBM + DF | TN/IL | 61.4 |
| 5 | NBM + DF | TN/IL | 63.3 |
| 6 | NBM + DF | TN/IL | 64.2 |
| 7 | NBM + sup* | FBS | 31 |

### Example 2: Activation with TNF-Alpha and/or IL-1-Beta, Alone or in Combination

Placental ASC expanded in NBM+supplement were activated in tissue culture dishes for 24 hours with 25 ng/mL of either TNF-alpha, IL-1 beta, or both cytokines combined. CM from the activated cells were tested for cytokine concentrations and BMM induction, relative to negative control (uninduced cells) and positive control (cells induced with 20% FBS). TNF-alpha and IL-1 beta combined were significantly more potent than either factor alone (Table 5). Robust activation with this combination of cytokines was consistently observed over several experiments, with several batches of ASC.

TABLE 5
Characteristics of cells activated with TNF-α, IL-1β, or both.

| Activating | | | | | |
|---|---|---|---|---|---|
| cytokine(s) | % BMM | % rel. potency | IL-8 | IL-6 | MCP |
| IL-1β | 13 | 35 | 2,859 | 1,440 | 2,218 |
| TNF-α | 8 | 23 | 2,747 | 1,434 | 5,339 |
| TNF-α + IL-1β | 21 | 60 | 16,403 | 15,920 | 11,794 |
| Pos. control | 5 | 13 | 52 | 264 | 604 |
| Neg. control | 6 | 16 | 26 | 70 | 284 |

### Example 3: Activation for/with Varied Times and Concentrations

Placental ASC expanded in NBM+supplement were activated in tissue culture dishes for 24 or 96 hours (refreshing the cytokines after 48 hours) with 5, 10, or 25 ng/mL of TNF-alpha+IL-1 beta, and CM were tested for cytokine concentrations and BMM induction. Activation was successful throughout the range of times and concentrations (Table 6).

TABLE 6
Characteristics of cells activated for/with varied times and concentrations.

| Cytokine conc./activation time | % BMM | IL-8 | IL-6 | MCP |
|---|---|---|---|---|
| 5/24 | 37 | 16,511 | 30,320 | 24,036 |
| 5/96 | 33 | 6,955 | 3,531 | 32,132 |
| 10/24 | 35 | 23,812 | 45,478 | 22,282 |
| 10/96 | 38 | 11,936 | 7,436 | 32,055 |
| 25/24 | 44 | 25,294 | 41,304 | 29,272 |
| 25/96 | 31 | 6,812 | 4,005 | 26,280 |

### Example 4: Inclusion of IL-4 as an Activation Factor

Following expansion, placental ASC were activated in tissue culture flasks for 24 hours, with 5, 10, or 25 ng/mL of TNF-alpha+IL-1 beta; or 25 ng/mL of TNF-alpha+IL-1 beta+10 ng/mL of IL-4. CM were tested for cytokine concentrations and BMM induction. IL-4 conferred enhanced activation (Table 7).

TABLE 7
Characteristics of cells activated with or without IL-4.

| Cytokine conc: TNF-a/IL-lb/IL-4 | % BMM | IL-8 | IL-6 |
|---|---|---|---|
| 5/5/0 | 35 | 27,368 | 61,568 |
| 10/10/0 | 35 | 42,598 | 102,643 |
| 25/25/0 | 36 | 44,165 | 93,019 |
| 25/25/10 | 36 | 80,552 | 148,474 |

### Example 5: Protein Arrays of Cytokine-Activated Placental ASC Methods

RayBio® Protein Arrays (Cat # AAH-CYT-G5-8) were used according to the manufacturer's instructions.

### Results

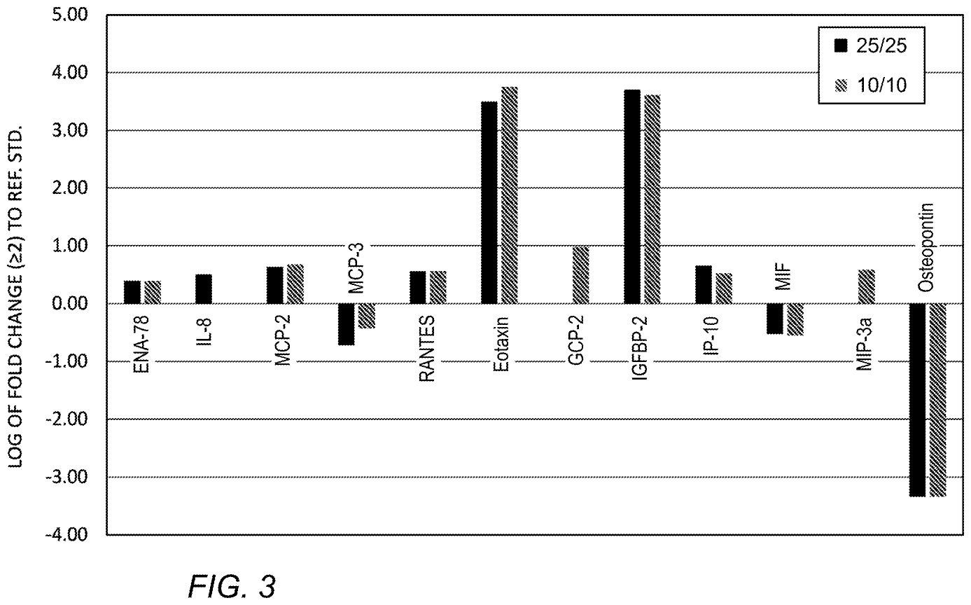

A protein array was performed on placental ASC expanded in Nutristem+defined factors or supplement, then activated in a bioreactor with 10 or 25 ng/mL each of TNF-alpha+IL-1 beta. Placental ASC expanded in NBM+supplement, then activated with 20% FBS, were used as a reference standard. The serum-free ASC exhibited a several thousand-fold increase over the reference standard in secretion of Eotaxin and IGFBP-2 (FIG. 3).

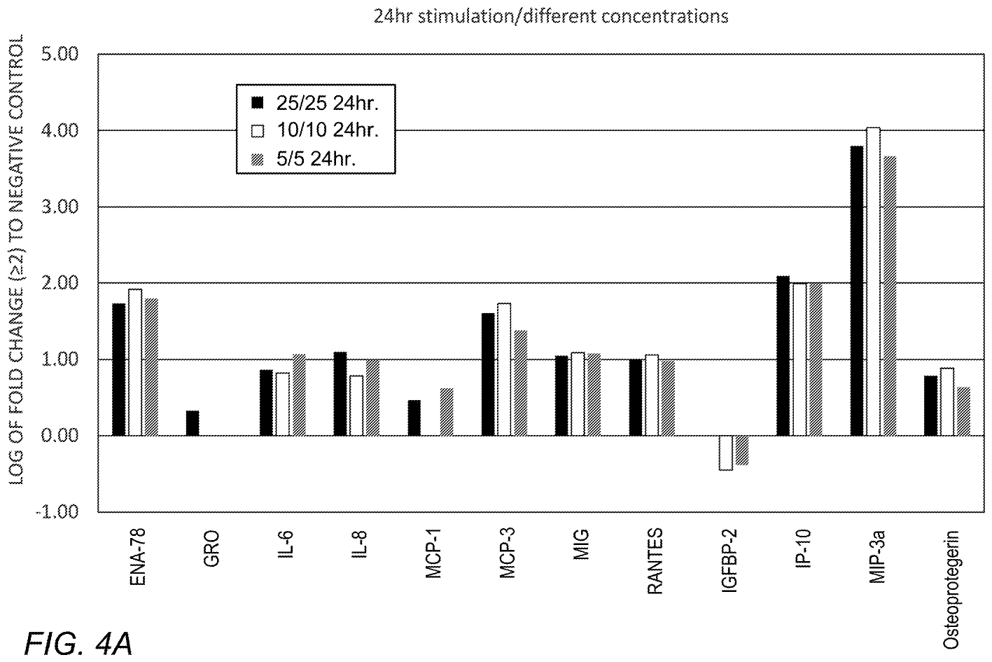

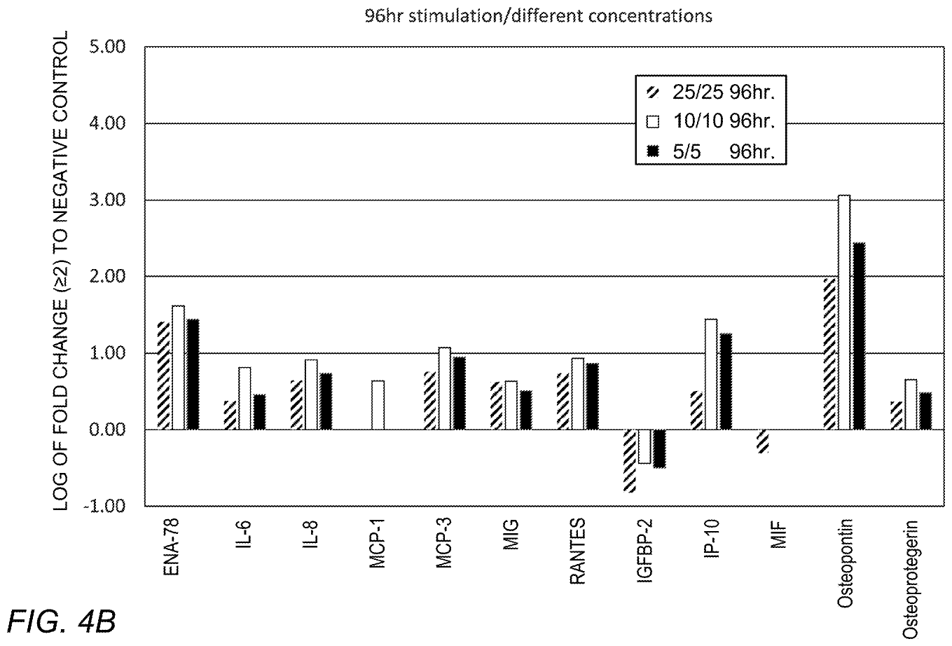

Another protein array was performed on placental ASC that were activated, following expansion, in tissue culture flasks for 24 or 96 hours, with 5, 10, or 25 ng/mL each of TNF-alpha+IL-1 beta. In this case, expression was compared to the negative control, unstimulated cells at the same passage number. Increased protein expression occurred throughout the range of time and cytokine concentrations, with a general trend towards higher protein expression after 24 hrs. vs. 96 hrs. of stimulation. MIP-3a expression exhibited an over 1,000-fold increase after 24 hr stimulation, but less than a 2-fold change at 96 hr, while Osteopontin showed the reverse pattern (FIGS. 4A-B).

### Example 6: Use of Expanded Placental Cells for Treating Ars

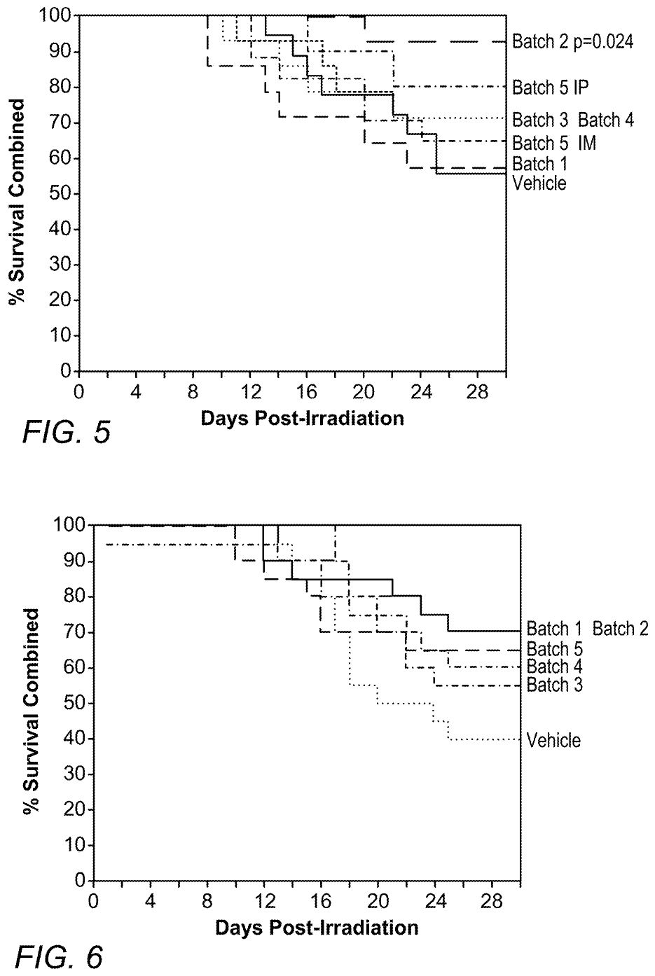

Placental ASC expanded by the described methods were tested in an animal model of acute radiation syndrome (ARS). C57BL/6 mice (n=14) were irradiated with 872 cGy, corresponding to the LD 70/30 (causing 70% mortality within 30 days) on day 0, and then were treated on days 1 and 5 with intramuscular (IM) injections of 2 million fetal placental cells, obtained by the described two-step expansion protocols, with the second medium containing activating factors (batches 1-3, with the same group numbers) or serum (batches 4-5, with the same group numbers). The average dose was 2 million cells, which was weight adjusted for individual mice. In another group (“Batch 5 IP”), Batch 5 was administered intraperitoneally instead. Vehicle (PlasmaLyte 148; Group 7) served as the negative control. A survival-enhancing effect was seen in nearly every group (FIG. 5).

A similar experiment was performed to confirm the hematopoietic benefits of the expanded fetal/placental ASC (n=18), again with fetal placental cells, obtained by the described two-step expansion protocols utilizing activating factors (batches/groups 1-3) or serum (batches/groups 4-5, with the same group numbers). None of groups 1-3 was produced by the expansion protocol used to produce batch 1 in the previous experiment. A survival-enhancing effect was seen in every group (FIG. 6).

In two additional experiments, IM-injected fetal/placental ASC administered on days minus 1 and 3 exhibited even greater protective effects from ARS, enhancing 30-day survival to 88% vs. 40% for controls, or 74% vs. 9% (n=24 for both experiments).

### Example 7: Use of Expanded Placental Cells for Facilitating HSC Engraftment

Expanded placental ASC are tested in an animal model of HSC engraftment, for example a murine model that measures engraftment of human cells. Non-limiting examples of such models are described in Wiekmeijer A S et al. and the references cited therein.

### Example 8: Use of Expanded Placental Cells in Treating Incomplete Engraftment

Subjects with delayed or incomplete engraftment, as defined in Trébéden-Negre H et al and the references cited therein, are administered expanded placental cells, typically between 1-24 months after the transplant. In other experiments, the cells may be administered together with an additional transplant. Amelioration of the disorder is evidence of therapeutic efficacy.

### Example 9: Use of Expanded Placental Cells in Enhancing Hematopoiesis Following an RIC Hsc Transplant

Expanded placental cells are tested in an animal model of hematopoiesis following a reduced intensity conditioning (RIC) HSC transplant, for example as described in Chandrasekaran D et al, Koyama M et al, and the references cited therein. In still other experiments, human subjects having received an RIC HSC are administered the described cells, for example as a single infusion within 14 days of receiving the transplant, or as 2-5 separate infusions over a 1-4 month period, within 3 months of the transplant. Amelioration of the disorder is evidence of therapeutic efficacy.

### Example 10: Use of Expanded Placental Cells in Treating MDS

Expanded placental ASC are tested in an animal model of myelodysplastic syndrome (MDS), for example as described in Inoue D et al, Li X et al, and the references cited therein. In other experiments, human subjects with MDS are administered the described cells. Amelioration of the disorder is evidence of therapeutic efficacy. In still other experiments, the effect of the cells on the incidence of acute myeloid leukemia (AML) is assessed.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace alternatives, modifications and variations that fall within the spirit and broad scope of the claims and description. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the invention.

### REFERENCES

### Additional References are Cited in Text

Aggarwal B B et al, Historical perspectives on tumor necrosis factor and its superfamily: 25 years later, a golden journey. Blood. 2012 Jan. 19; 119(3):651-65.
Bian Z M et al, IL-4 potentiates IL-1beta- and TNF-alpha-stimulated IL-8 and MCP-1 protein production in human retinal pigment epithelial cells. Curr Eye Res. 1999 May; 18(5):349-57.
Carrero R et al, ILO induces mesenchymal stem cells migration and leucocyte chemotaxis through NF-κB. Stem Cell Rev. 2012 September; 8(3):905-16.
Chandrasekaran D et al, Modeling promising nonmyeloablative conditioning regimens in nonhuman primates. Hum Gene Ther. 2014 December; 25(12):1013-22.
Chase L G et al, Development and Characterization of a Clinically Compliant Xeno-Free Culture Medium in Good Manufacturing Practice for Human Multipotent Mesenchymal Stem Cells. Stem Cells Transl Med. 2012 October; 1(10): 750-758.
Clayton A et al, Analysis of antigen presenting cell derived exosomes, based on immuno-magnetic isolation and flow cytometry. J Immunol Methods. 2001; 247(1-2):163-74.
Coffin J D et al, Fibroblast Growth Factor 2 and Its Receptors in Bone Biology and Disease. J Endocr Soc. 2018 May 28; 2(7):657-671.
Crescitelli R et al, Distinct RNA profiles in subpopulations of extracellular vesicles: apoptotic bodies, microvesicles and exosomes. J Extracell Vesicles. 2013 Sep. 12; 2.
Davis and Pennypacker, The role of the leukemia inhibitory factor receptor in neuroprotective signaling. Pharmacol Ther. 2018 March; 183:50-57
Derynck and Budi, Specificity, versatility, and control of TGF-β family signaling. Sci Signal. 2019 Feb. 26; 12(570). pii: eaav5183. doi: 10.1126/scisignal.aav5183.
Docheva D et al, Human mesenchymal stem cells in contact with their environment: surface characteristics and the integrin system. J Cell Mol Med. 2007 January; 11(1): 21-38.
Dominici et al. Minimal criteria for defining multipotent mesenchymal stromal cells. The International Society for Cellular Therapy position statement. Cytotherapy. 2006; 8(4):315-7.
Dulbecco and Freeman (1959) Plaque production by the polyoma virus. Virology 8(3):396-39.
Fang C Y et al, Long-term growth comparison studies of FBS and FBS alternatives in six head and neck cell lines. PLoS One. 2017 Jun. 7; 12(6):e0178960.
Heldin and Moustakas, Signaling Receptors for TGF-β Family Members. Cold Spring Harb Perspect Biol. 2016 Aug. 1; 8(8).
Imamura. Physiological Functions and Underlying Mechanisms of Fibroblast Growth Factor (FGF) Family Members: Recent Findings and Implications for Their Pharmacological Application. Biological and Pharmaceutical Bulletin, Vol. 37(7):1081-1089 (2014).
Inoue D et al, Myelodysplastic syndromes are induced by histone methylation-altering ASXL1 mutations. J Clin Invest. 2013 November; 123(11):4627-40.
Itoh N. et al. (2011) Fibroblast growth factors: From molecular evolution to roles in development, metabolism and disease. J. Biochem., 149, 121-130.
Kazlauskas A, PDGFs and their receptors. Gene. 2017 May 30; 614:1-7.
Kinzebach S and Bieback K. Expansion of Mesenchymal Stem/Stromal cells under xenogenic-free culture conditions. Adv Biochem Eng Biotechnol. 2013; 129:33-57.
Koyama M, Expansion of donor-reactive host T cells in primary graft failure after allogeneic hematopoietic SCT following reduced-intensity conditioning. Bone Marrow Transplant. 2014; 49(1):110-5.
Lin and Leonard, The Common Cytokine Receptor y Chain Family of Cytokines. Cold Spring Harb Perspect Biol. 2018 Sep. 4; 10(9).
Malik and Kanneganti, Function and regulation of IL-1α in inflammatory diseases and cancer. Immunol Rev. 2018 January; 281(1):124-137. doi: 10.1111/imr.12615.
Mathias R A et al, Isolation of extracellular membranous vesicles for proteomic analysis.
Mimura et al, Growth factor-defined culture medium for human mesenchymal stem cells. Int. J. Dev. Biol. 55: 181-187 (2011).
Modrowski D et al., Involvement of interleukin 1 and tumour necrosis factor alpha as endogenous growth factors in human osteoblastic cells. Cytokine 1995 October; 7(7):720-6.
Ng F et al, PDGF, TGF-beta, and FGF signaling is important for differentiation and growth of mesenchymal stem cells (MSCs): transcriptional profiling can identify markers and signaling pathways important in differentiation of MSCs into adipogenic, chondrogenic, and osteogenic lineages. Blood. 2008 Jul. 15; 112(2):295-307.
Nicola and Babon, Leukemia inhibitory factor (LIF). Cytokine Growth Factor Rev. 2015 October; 26(5):533-44.
Rajaraman G et al, Optimization and scale-up culture of human endometrial multipotent mesenchymal stromal cells: potential for clinical application. Tissue Eng Part C Methods. 2013 January; 19(1):80-92.
Sullivan C B et al, TNFα and IL-1β influence the differentiation and migration of murine MSCs independently of the NF-κB pathway. Stem Cell Res Ther. 2014 Aug. 27; 5(4):104.
Trébéden-Negre H et al, Delayed recovery after autologous peripheral hematopoietic cell transplantation: potential effect of a high number of total nucleated cells in the graft. Transfusion. 2010 December; 50(12):2649-59.
van den Dolder J et al, Platelet-rich plasma: quantification of growth factor levels and the effect on growth and differentiation of rat bone marrow cells. Tissue Eng. 2006 November; 12(11):3067-73.
Wiekmeijer A S et al. Sustained Engraftment of Cryopreserved Human Bone Marrow CD34(+) Cells in Young Adult NSG Mice. Biores Open Access. 2014; 3(3):110-6.

What is claimed is:

1. A method of expanding a population of adherent stromal cells (ASC), comprising:
a. incubating said ASC population in a first medium, wherein (i) said medium contains less than 5% animal serum; and (b) said medium comprises a component selected from Transferrin, insulin FGF, TGF-beta, PDGF, and EGF, thereby obtaining a first expanded ASC population; and
b. incubating the first expanded cell population in a second medium, wherein said second medium contains less than 5% animal serum, and said second medium comprises one or more activating components,
thereby expanding a population of ASC.

2. (canceled)

3. The method of claim 1, wherein said first medium does not contain animal serum.

4. The method of claim 1, wherein said first medium comprises an FGF selected front FGF-1, FGF-2, and an FGF-1-FGF-2 chimera.

5. The method of claim 4, wherein said first medium further comprises TGF-beta.

6. The method of claim 5, wherein said first medium further comprises PDGF.

7. The method of claim 5, wherein said medium further comprises a glucocorticoid.

8. The method of claim 1, wherein said ASC is incubated in said first medium for at least 10 population doublings.

9. (canceled)

10. The method of claim 1, wherein said second medium does not contain animal serum.

11. The method of claim 1, wherein said second medium further comprises a component selected from Transferrin, Insulin, an FGF, TGF-beta, PDGF, and EGF.

12. The method of claim 1, wherein said ASC originate from placenta tissue.

13. The method of claim 1, wherein said ASC originate from adipose tissue or bone marrow.

14. (canceled)

15. The method of claim 1, wherein over 90% of said expanded ASC population express a marker selected from the group consisting of CD73, CD90, CD29 and CD105.

16. The method of claim 15, wherein over 90% of said expanded ASC population do not express a marker selected from the group consisting of CD3, CD4, CD34, CD39, and CD106.

17-18. (canceled)

19. The method of claim 16, wherein more than 50% of said expanded ASC population express CD141.

20. (canceled)

21. The method of claim 16, wherein more than 50% of said expanded ASC population express HLA-A2.

22. The method of claim 15, wherein said ASC do not express a marker selected from the group consisting of CD3, CD4, CM11b, CD14, CD19, and CD34.

23. Cells obtained by the method of claim 1.

24. A pharmaceutical composition comprising the cells of claim 23.

25. A bioreactor comprising the cells of claim 23.

26. The bioreactor of claim 25, wherein said bioreactor further comprises a synthetic three-dimensional growth substrate.
